(19) United States
(12) Patent Application Publication
(10) Pub. No.: US 2014/0199298 A1
(43) Pub. Date: Jul. 17, 2014

(54) COMBINATION TREATMENT OF CANCER COMPRISING EGFR/HER2 INHIBITORS

(71) Applicants: Flavio SOLCA, Vienna (AT); Andree AMELSBERG, Southbury, CT (US); Gerd STEHLE, Ehingen (DE); Jacobus C.A. VAN MEEL, Moedling (AT); Anke BAUM, Hinterbruehl (AT)

(72) Inventors: Flavio SOLCA, Vienna (AT); Andree AMELSBERG, Southbury, CT (US); Gerd STEHLE, Ehingen (DE); Jacobus C.A. VAN MEEL, Moedling (AT); Anke BAUM, Hinterbruehl (AT)

(73) Assignee: BOEHRINGER INGELHEIM INTERNATIONAL GMBH, Ingelheim am Rhein (DE)

(21) Appl. No.: 14/160,700

(22) Filed: Jan. 22, 2014

Related U.S. Application Data
(63) Continuation of application No. 12/093,322, filed on Sep. 9, 2008.

(30) Foreign Application Priority Data
Nov. 11, 2005 (EP) 05110669.8

(51) Int. Cl. C07D 405/12; A61K 31/517; A61K 39/395

(52) U.S. Cl. CPC C07D 405/12; A61K 39/39558; A61K 31/517
USPC 424/133.1

(57) ABSTRACT

The invention relates to a therapy of cancer comprising co-administration to a person in need of such treatment and/or co-treatment of a person in need of such treatment with effective amounts of:
(1) a compound 1 of formula (I)

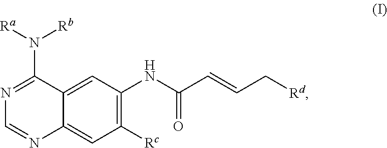

wherein the groups R^{a} to R^{d} have the meanings given in the claims and specification; and
(2) at least a further chemotherapeutic agent 2;
   optionally in combination with radiotherapy, radio-immunotherapy and/or tumour resection by surgery, furthermore, the invention relates to corresponding medicaments and the preparation thereof.

The invention relates to a therapy of cancer comprising co-administration to a person in need of such treatment and/or co-treatment of a person in need of such treatment with effective amounts of:
(1) a compound 1 of formula (I)

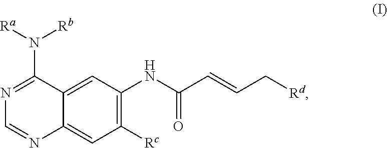

to wherein the groups R^{a} to R^{d} have the meanings given in the claims and specification; and
(2) at least a further chemotherapeutic agent 2;
   optionally in combination with radiotherapy, radio-immunotherapy and/or tumour resection by surgery.

### BACKGROUND OF THE INVENTION

Compounds of formula (I) are disclosed in WO 02/50043, WO 2004/074263 and WO 2005/037824 as dual inhibitors of erbb1 receptor (EGFR) and erbB2 (Her2/neu) receptor tyrosine kinases, suitable for the treatment of e.g. benign or malignant tumours, particularly tumours of epithelial and neuroepithelial origin, metastasisation and the abnormal proliferation of vascular endothelial cells (neoangiogenesis), for treating diseases of the airways and lungs which are accompanied by increased or altered production of mucus caused by stimulation by tyrosine kinases, as well as for treating diseases of the gastrointestinal tract and bile duct and gall bladder which are associated with disrupted activity of the tyrosine kinases. The disclosure of WO 02/50043, WO 2004/074263 and WO 2005/037824 includes preparation as well as pharmaceutical formulations of the compounds and is incorporated by reference regarding these aspects. Furthermore, it is known for treatment of tumour diseases that the compounds may be used in monotherapy or in conjunction with other anti-tumour therapeutic agents, for example in combination with topoisomerase inhibitors (e.g. etoposide), mitosis inhibitors (e.g. vinblastine), compounds which interact with nucleic acids (e.g. cis-platin, cyclophosphamide, adriamycin), hormone antagonists (e.g. tamoxifen), inhibitors of metabolic processes (e.g. 5-FU etc.), cytokines (e.g. interferons) or antibodies. Treatment of tumour diseases with the combination of the VEGFR inhibitor 3-Z-[1-(4-(N-((4-methyl-piperazin-1-yl)-methylcarbonyl)-N-methyl-amino)-anilino)-1-phenyl-methylene]-6-methoxycarbonyl-2-indolinone and one of the dual EGFR/HER2 inhibitors 4-[(3-chloro-4-fluorophenyl)amino]-6-{[4-(N,N-dimethylamino)-1-oxo-2-buten-1-yl]amino}-7-((S)-tetrahydrofuran-3-yloxy)-quinazoline or 4-[(3-chloro-4-fluoro-phenyl)amino]-6-{[4-(homomorpholin-4-yl)-1-oxo-2-buten-1-yl]amino}-7-[(S)-(tetrahydrofuran-3-yl)oxy]-quinazoline are disclosed in WO 2004/096224.

For the treatment of diseases of oncological nature, a large number of chemotherapeutic, immunotherapeutic or immunomodulatory, antiangiogenic or hormonal agents have already been suggested, which can be used as monotherapy (treatment with one agent) or as combination therapy (simultaneous, separate or sequential treatment with more than one agent) and/or which may be combined with radiotherapy or radio-immunotherapy. In this respect, chemotherapeutic agent means a naturally occurring, semi-synthetic or synthetic chemical compound which, alone or via further activation, for example with radiations in the case of radio-immunotherapy, inhibits or kills growing cells, and which can be used or is approved for use in the treatment of diseases of oncological nature, which are commonly also denominated as cancers. In the literature, these agents are generally classified according to their mechanism of action. In this matter, reference can be made, for example, to the classification made in “Cancer Chemotherapeutic Agents”, American Chemical Society, 1995, W. O. Foye Ed.

The efficacy of chemotherapeutic agents can be improved by using combination therapies with other chemotherapeutic, immunotherapeutic, immunomodulatory, antiangiogenic or hormonal compounds. Combination therapies constitute the gold standard in many settings of cancer therapy.

Even if the concept of combining several therapeutic agents or therapies already has been suggested, and although various combination therapies are under investigation and in clinical trials, there is still a need for new and efficient therapeutic compositions for the treatment of cancer diseases, which show advantages over standard therapies. It is the purpose of the present invention to provide a combination therapy with the dual inhibitors of formula (I) for the treatment of various cancer diseases.

### SUMMARY OF THE INVENTION

It has been found that a combination therapy for treatment of various cancer diseases, especially of the specific cancer-subindications mentioned hereinafter, comprising co-administration to a patient and/or co-treatment of a patient with effective amounts of:
(1) a compound 1 of formula (I)

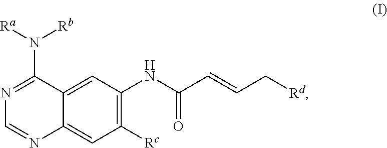

is wherein the groups R^{a} to R^{d} have the meanings given in the claims and specification; and
(2) at least a further chemotherapeutic agent 2;
   optionally in combination with radiotherapy, radio-immunotherapy and/or tumour resection by surgery,
   provides unexpected advantages, e.g. superior efficacy based on additive or synergistic effects and/or improved tolerability and reduced side effects of the treatment by the patient due, for example, to the administration of lower doses of the therapeutic agents involved reduced side effects.

Any reference to a compound 1 of formula (I) in connection with the invention should be understood to include the tautomers, racemates, enantiomers and diastereomers thereof, if any, the mixtures thereof as well as the pharmacologically acceptable acid addition salts, solvates, hydrates, polymorphs, physiologically functional derivatives or metabolites, or prodrugs thereof.

The expression “patient” relates to a human or non-human mammalian patient suffering from cancer and thus in need of such treatment, preferably the patient is a human person. Furthermore, the expression “patient” should be understood to include such cancer patients carrying tumors with wild-type EGF receptor as well as pre-selected cancer patients with tumors harboring activating EGFR mutations. These can be located in the tyrosine kinase domain of the EGF receptor such as for instance the L858R or L861 point mutations in the activation loop (exon 21), or in-frame deletion/insertion mutations in the ELREA sequence (exon 19), or substitutions in G719 situated in the nucleotide binding loop (exon 18). Additional activating mutations have been reported in the extracellular domain of the EGF receptor in various indications (e.g. EGFR vIII displaying exon 2-7 deletions). Other mutations such as the T790M point mutation in exon 20 as well as certain exon 20 insertions (e.g. D770_N771insNPG) which confer resistance to particular drugs should also be included, as well as double mutants such as the combined L858R/T790M mutation or the exon-19-del/T790M. The expression “patient” should be understood to include also such cancer patients carrying tumors with wild-type HER2 receptor as well as pre-selected cancer patients with tumors harboring activating HER2 mutations, e.g. M774_A775insAYVM.

The indication “cancer” as used in the context of the invention is to be understood in a most general sense as a disease characterized by inappropriate cellular proliferation, migration, apoptosis or angiogenesis, preferably by inappropriate cellular proliferation. Inappropriate cell proliferation means cellular proliferation resulting from inappropriate cell growth, from excessive cell division, from cell division at an accelerated rate and/or from inappropriate cell survival.

The expression “chemotherapeutic agent 2” refers to any chemotherapeutic, immunotherapeutic or immunomodulatory, antiangiogenic, hormonal or naturally occurring, semi-synthetic or synthetic therapeutic agent 2 known or suitable for tumour therapy. Any reference to a chemotherapeutic agent 2 in connection with the invention should be understood to include the tautomers, racemates, enantiomers and diastereomers thereof, if any, the mixtures thereof as well as the pharmacologically acceptable acid addition salts, solvates, hydrates, polymorphs, physiologically functional derivatives or metabolites, or prodrugs thereof.

“Radiotherapy” means administering ionizing radiation to the patient, as conventionally used in cancer therapy. Radiotherapy may be applied before, in parallel or after co-treatment by administration of the actives 1 and 2.

“Tumour resection by surgery” is one standard option in cancer therapy and may be applied before or after co-treatment by administration of the actives 1 and 2.

A first aspect of the present invention therefore is a method of treating cancer, preferably the specific cancer-subindications referred to hereinafter, said method comprising co-administration to a person in need of such treatment and/or co-treatment of a person in need of such treatment with effective amounts of:
(1) a compound 1 of formula (I); and
(2) at least a further chemotherapeutic agent 2;
   optionally in combination with radiotherapy, radio-immunotherapy and/or tumour resection by surgery.

A second aspect of the present invention relates to a pharmaceutical composition for the treatment of cancer comprising effective amounts of:
(1) a compound 1 of formula (I); and
(2) at least a further chemotherapeutic agent 2;
   optionally in combination with one or more pharmaceutically acceptable excipients, and optionally adapted for a co-treatment with radiotherapy or radio-immunotherapy, in the form of a combined preparation for simultaneous, separate or sequential use in the treatment of diseases involving cell proliferation, migration or apoptosis of cancer cells, or angiogenesis, preferably involving cell proliferation or apoptosis of cancer cells.

A third aspect of the present invention is directed to the use of a compound 1 of formula (I) for the manufacture of a pharmaceutical composition for the treatment of cancer, preferably for the treatment of the specific cancer-subindications referred to hereinafter, comprising effective amounts of:
(1) a compound 1 of formula (I); and
(2) at least a further chemotherapeutic agent 2;
   optionally in combination with one or more pharmaceutically acceptable excipients, and optionally adapted for a co-treatment with radiotherapy or radio-immunotherapy, in the form of a combined preparation for simultaneous, separate or sequential use in the treatment of diseases involving cell proliferation, migration or apoptosis of cancer cells, or angiogenesis, preferably involving cell proliferation or apoptosis of cancer cells.

The expression “a pharmaceutical composition for the treatment of cancer” should be understood interchangeable with “a medicament for the treatment of cancer”.

### BRIEF DESCRIPTION OF THE DRAWINGS

FIG. 1 is a graphical depiction of results of an experiment comparing daily treatment of gastric cancer N87 xenografts in mice with BIBW2992 alone (15 mg/kg), once weekly cis-platin alone (5 mg/kg) and the combination of BIBW2992/cis-platin (15 mg/kg/(5 mg/kg);

FIG. 2 is a graphical depiction of results of an experiment comparing treatment of ovarian cancer SKOV-3 xenografts in mice with daily administration of BIBW2992 alone (15 mg/kg), once weekly docetaxel alone (20 mg/kg) and the combination of BIBW2992/docetaxel (15 mg/kg/(20 mg/kg);

FIG. 3 is a graphical depiction of results of an experiment comparing daily treatment of ovarian cancer SKOV-3 xenografts in mice with BIBW2992 alone (15 mg/kg), docetaxel alone (15 mg/kg) once weekly and the combination of BIBW2992/docetaxel (15 mg/kg/(15 mg/kg);

FIG. 4 is a graphical depiction of results of an experiment comparing daily treatment of ovarian cancer SKOV-3 xenografts in mice with BIBW2992 alone (10 mg/kg), once weekly treatment with docetaxel alone (10 mg/kg) and the combination of BIBW2992/docetaxel (10 mg/kg/(10 mg/kg);

FIG. 5 is a graphical depiction of results of an experiment comparing treatment of ovarian cancer SKOV-3 xenografts in mice with BIBW2992 alone (35 mg/kg), administered twice weekly on two consecutive days, docetaxel alone (10 mg/kg), given once weekly and the pulsatile combination of BIBW2992 and docetaxel;

FIGS. 6A and 6B are graphical depictions of results of an experiment comparing daily treatment (day 1-11) of MDA-453 breast xenografts in mice with BIBW2992 alone (two dosages of: 15 mg/kg (FIG. 6A); 10 mg/kg (FIG. 6B)), doxorubicin alone (6 mg/kg) once weekly (day 1+8) and the combination of BIBW2992/doxorubicin (two dosages of: 15 mg/kg/6 mg/kg (FIG. 6A); 10 mg/kg/6 mg/kg (FIG. 6B)); and

FIG. 7 is a graphical depiction of inhibition of 5-FU combined with BIBW2992 in anchorage independent SKOV-3 cell assay.

### DETAILED DESCRIPTION OF THE INVENTION

In a first embodiment (1), with regard to the first, second and third aspect of the invention, formula (I)

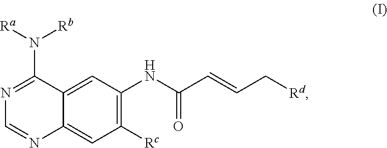

is defined to encompass those compounds 1 wherein
R^{a} denotes a benzyl, 1-phenylethyl or 3-chloro-4-fluorophenyl group,
R^{b} denotes a hydrogen atom or a C₁₋₄-alkyl group,
R^{c} denotes a cyclopropylmethoxy, cyclobutyloxy, cyclopentyloxy, tetrahydrofuran-3-yl-oxy, tetrahydrofuran-2-yl-methoxy, tetrahydrofuran-3-yl-methoxy, tetrahydropyran-4-yl-oxy or tetrahydropyran-4-yl-methoxy group,
R^{d} denotes a dimethylamino, N-cyclopropyl-N-methyl-amino, N-cyclopropylmethyl-N-methyl-amino, N-ethyl-N-methyl-amino, N,N-diethylamino, N-isopropyl-N-methyl-amino, N-(2-methoxyethyl)-N-methyl-amino, N-(1-methoxy-2-propyl)-N-methyl-amino, N-(3-methoxypropyl)-N-methyl-amino, pyrrolidino, 2-methylpyrrolidino, 2-(methoxymethyl)-pyrrolidino, morpholino, (1S,4S)-2-oxa-5-aza-bicyclo[2.2.1]hept-5-yl, (1R,4R)-2-oxa-5-aza-bicyclo[2.2.1]hept-5-yl, N-cyclopropyl-N-methyl-amino-, N-methyl-N-(tetrahydro furan-3-yl)-amino, N-methyl-N-(tetrahydrofuran-2-yl-methyl)-amino, N-methyl-N-(tetrahydrofuran-3-yl-methyl)-amino, N-methyl-N-(tetrahydropyran-4-yl)-amino or N-methyl-N-(tetrahydropyran-4-yl-methyl)-amino group, or a group of formula (II)

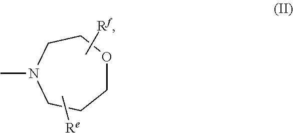

wherein R^{e} and R^{f}, which may be identical or different, in each case denote a is hydrogen atom or a C₁₋₃-alkyl group,
   subject to proviso (i) that if compound 1 is selected from
(d) 4-[(3-chloro-4-fluorophenyl)amino]-6-{[4-(N,N-dimethylamino)-1-oxo-2-buten-1-yl]amino}-7-((S)-tetrahydrofuran-3-yloxy)-quinazoline, and
(k) 4-[(3-chloro-4-fluoro-phenyl)amino]-6-{[4-(homomorpholin-4-yl)-1-oxo-2-buten-1-yl]amino}-7-[(S)-(tetrahydrofuran-3-yl)oxy]-quinazoline,
   the chemotherapeutic agent 2 is not 3-Z-[1-(4-(N-((4-methyl-piperazin-1-yl)-methylcarbonyl)-N-methyl-amino)-anilino)-1-phenyl-methylene]-6-methoxycarbonyl-2-indolinone.

Proviso (i) applies to any aspect and embodiment of the invention.

In a second embodiment (2), with regard to any aspect of the invention, formula (I)

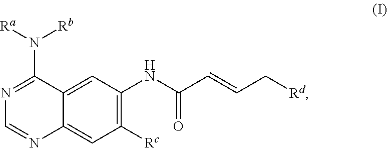

is defined to encompass those compounds 1 wherein
R^{a} denotes a 3-chloro-4-fluorophenyl group,
R^{b} denotes a hydrogen atom,
R^{c} denotes a cyclopropylmethoxy, cyclobutyloxy, cyclopentyloxy, tetrahydrofuran-3-yl-oxy, tetrahydrofuran-2-yl-methoxy, tetrahydrofuran-3-yl-methoxy, tetrahydropyran-4-yl-oxy or tetrahydropyran-4-yl-methoxy group,
R^{d} denotes a dimethylamino, N-cyclopropyl-N-methyl-amino, N-cyclopropylmethyl-N-methyl-amino, N-ethyl-N-methyl-amino, N,N-diethylamino, N-isopropyl-N-methyl-amino, N-(2-methoxyethyl)-N-methyl-amino, N-(1-methoxy-2-propyl)-N-methyl-amino, N-(3-methoxypropyl)-N-methyl-amino, pyrrolidino, 2-methylpyrrolidino, 2-(methoxymethyl)-pyrrolidino, morpholino, (1S,4S)-2-oxa-5-aza-bicyclo[2.2.1]hept-5-yl, (1R,4R)-2-oxa-5-aza-bicyclo[2.2.1]hept-5-yl, N-methyl-N-(tetrahydrofuran-3-yl)-amino, N-methyl-N-(tetrahydrofuran-2-yl-methyl)-amino, N-methyl-N-(tetrahydrofuran-3-yl-methyl)-amino, N-methyl-N-(tetrahydropyran-4-yl)-amino or N-methyl-N-(tetrahydropyran-4-yl-methyl)-amino group, or a group of formula (II)

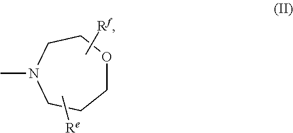

wherein R^{e} and R^{f} denote a hydrogen atom.

In a third embodiment (3), with regard to any aspect of the invention, formula (I)

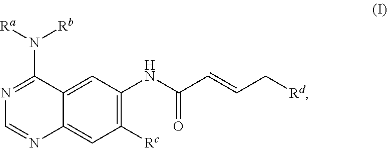

is defined to encompass those compounds 1 wherein
R^{a} denotes a 3-chloro-4-fluorophenyl group,
R^{b} denotes a hydrogen atom,
R^{c} denotes a tetrahydrofuran-3-yl-oxy, tetrahydrofuran-2-yl-methoxy, tetrahydrofuran-3-yl-methoxy, tetrahydropyran-4-yl-oxy or tetrahydropyran-4-yl-methoxy group,
R^{d} denotes a dimethylamino, N-cyclopropyl-N-methyl, N-ethyl-N-methyl-amino, N,N-diethylamino, N-isopropyl-N-methyl-amino, morpholino, (1S,4S)-2-oxa-5-aza-bicyclo-[2.2.1]hept-5-yl or (1R,4R)-2-oxa-5-aza-bicyclo[2.2.1]hept-5-yl, group, or a group of formula (II)

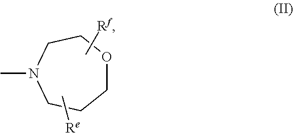

wherein R^{e} and R^{f} denote a hydrogen atom.

In a fourth embodiment (4), with regard to any aspect of the invention, formula (I)

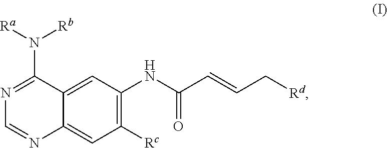

is defined to encompass those compounds 1 wherein
R^{a} denotes a 3-chloro-4-fluorophenyl group,
R^{b} denotes a hydrogen atom,
R^{c} denotes a tetrahydrofuran-3-yl-oxy, tetrahydrofuran-2-yl-methoxy or tetrahydrofuran-3-yl-methoxy group,
R^{d} denotes a dimethylamino group or a group of formula (II)

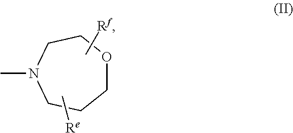

wherein R^{e} and R^{f}, denote a hydrogen atom.

In a fifth embodiment (5), with regard to any aspect of the invention, formula (I) is defined to encompass the compounds 1 selected from the group consisting of
(a) 4-[(3-chloro-4-fluorophenyl)amino]-6-{[4-(N,N-dimethylamino)-1-oxo-2-buten-1-yl]amino}-7-cyclobutyloxy-quinazoline,
(b) 4-[(3-chloro-4-fluorophenyl)amino]-6-{[4-(N,N-dimethylamino)-1-oxo-2-buten-1-yl]amino}-7-cyclopentyloxy-quinazoline,
(c) 4-[(3-chloro-4-fluorophenyl)amino]-6-{[4-(N,N-dimethylamino)-1-oxo-2-buten-1-yl]amino}-7-((R)-tetrahydrofuran-3-yloxy)-quinazoline,
(d) 4-[(3-chloro-4-fluorophenyl)amino]-6-{[4-(N,N-dimethylamino)-1-oxo-2-buten-1-yl]amino}-7-((S)-tetrahydrofuran-3-yloxy)-quinazoline,
(e) 4-[(3-chloro-4-fluorophenyl)amino]-6-{[4-(N,N-dimethylamino)-1-oxo-2-buten-1-yl]amino}-7-(tetrahydropyran-4-yloxy)-quinazoline,
(f) 4-[(3-chloro-4-fluorophenyl)amino]-6-{[4-(N,N-dimethylamino)-1-oxo-2-buten-1-yl]amino}-7-[(tetrahydrofuran-2-yl)methoxy]-quinazoline,
(g) 4-[(3-chloro-4-fluorophenyl)amino]-6-{[4-(N,N-dimethylamino)-1-oxo-2-buten-1-yl]amino}-7-[(tetrahydrofuran-3-yl)methoxy]-quinazoline,
(h) 4-[(R)-(1-phenyl-ethyl)amino]-6-{[4-(N,N-dimethylamino)-1-oxo-2-buten-1-yl]amino}-7-cyclopropylmethoxy-quinazoline,
(i) 4-[(3-chloro-4-fluorophenyl)amino]-6-{[4-(morpholin-4-yl)-1-oxo-2-buten-1-yl]amino}-7-[(tetrahydrofuran-2-yl)methoxy]-quinazoline,
(j) 4-[(3-chloro-4-fluorophenyl)amino]-6-{[4-(N,N-dimethylamino)-1-oxo-2-buten-1-yl]amino}-7-[(S)-(tetrahydrofuran-2-yl)methoxy]-quinazoline,
(k) 4-[(3-chloro-4-fluoro-phenyl)amino]-6-{[4-(homomorpholin-4-yl)-1-oxo-2-buten-1-yl]amino}-7-[(S)-(tetrahydrofuran-3-yl)oxy]-quinazoline,
(l) 4-[(3-chloro-4-fluoro-phenyl)amino]-6-{[4-(N-ethyl-N-methyl-amino)-1-oxo-2-buten-1-yl]amino}-7-[(S)-(tetrahydrofuran-3-yl)oxy]-quinazoline,
(m) 4-[(3-chloro-4-fluoro-phenyl)amino]-6-{[4-(N-isopropyl-N-methyl-amino)-1-oxo-2-buten-1-yl]amino}-7-[(S)-(tetrahydrofuran-3-yl)oxy]-quinazoline,
(n) 4-[(3-chloro-4-fluoro-phenyl)amino]-6-{[4-(N-cyclopropyl-N-methyl-amino)-1-oxo-2-buten-1-yl]amino}-7-cyclopentyloxy-quinazoline,
(o) 4-[(3-chloro-4-fluoro-phenyl)amino]-6-{[4-(N,N-diethyl-amino)-1-oxo-2-buten-1-yl]amino}-7-cyclopropylmethoxy-quinazoline,
(p) 4-[(3-chloro-4-fluoro-phenyl)amino]-6-{[4-((1S,4S)-2-oxa-5-aza-bicyclo[2.2.1]-hept-5-yl)-1-oxo-2-buten-1-yl]amino}-7-[(S)-(tetrahydrofuran-3-yl)oxy]-quinazoline,
(q) 4-[(3-chloro-4-fluoro-phenyl)amino]-6-{[4-((1R,4R)-2-oxa-5-aza-bicyclo[2.2.1]-hept-5-yl)-1-oxo-2-buten-1-yl]amino}-7-[(S)-(tetrahydrofuran-3-yl)oxy]-quinazoline and
(r) 4-[(3-chloro-4-fluoro-phenyl)amino]-6-{[4-(dimethylamino)-1-oxo-2-buten-1-yl]amino}-7-cyclopropylmethoxy-quinazoline.

In a sixth embodiment (6), with regard to any aspect of the invention, the compounds 1 of formula (I) are selected from the group consisting of
(d) 4-[(3-chloro-4-fluorophenyl)amino]-6-{[4-(N,N-dimethylamino)-1-oxo-2-buten-1-yl]amino}-7-((S)-tetrahydrofuran-3-yloxy)-quinazoline,

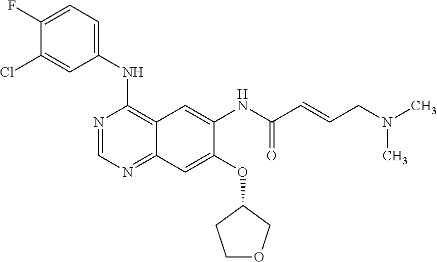 [SMILES: CN(C)C/C=C/C(=O)Nc1cc2c(Nc3ccc(F)c(Cl)c3)ncnc2cc1O[C@H]1CCOC1]

(k) 4-[(3-chloro-4-fluoro-phenyl)amino]-6-{[4-(homomorpholin-4-yl)-1-oxo-2-buten-1-yl]amino}-7-[(S)-(tetrahydrofuran-3-yl)oxy]-quinazoline,
   the dimaleate salt of compound (d) being especially preferred:
(d′) 4-[(3-chloro-4-fluorophenyl)amino]-6-{[4-(N,N-dimethylamino)-1-oxo-2-buten-1-yl]amino}-7-((S)-tetrahydrofuran-3-yloxy)-quinazoline dimaleate.

Within the meaning of the present invention, the following classes (7) of chemotherapeutic agents 2 are especially of interest, although not representing a limitation:
Synthetic small molecule VEGF receptor antagonists
Small molecule growth factor (GF) receptor antagonists
Inhibitors of the EGF receptor and/or HER2 receptors and/or VEGF receptor and/or integrin receptors or any other protein tyrosine kinase receptors, which are not classified under the synthetic small-molecules
Small molecule Polo-like kinase-1 (PLK-1) inhibitors
Small molecule inhibitors of the Ras/Raf/MAPK or PI3K/AKT pathways or any other serine/threonine kinases.
Inhibitors of the Ras/Raf/MAPK or PI3K/AKT pathways or any other serine/threonine kinases, which are not classified under the synthetic small-molecules
Inhibitors directed to EGF receptor and/or VEGF receptor and/or integrin receptors or any other protein tyrosine kinase receptors, which are synthetically manufactured antibodies, antibody fragments or fusion proteins
Inhibitors directed to circulating VEGF, which are synthetically manufactured antibodies, antibody fragments or fusion proteins
Compounds which interact with nucleic acids and which are classified as alkylating agents or platinum compounds
Compounds which interact with nucleic acids and which are classified as anthracyclines, as DNA intercalators or as DNA cross-linking agents
Anti-metabolites
Naturally occurring, semi-synthetic or synthetic bleomycin type antibiotics (BLM-group antibiotics)
Inhibitors of DNA transcribing enzymes, especially topoisomerase I or topoisomerase II inhibitors
Chromatin modifying agents
Mitosis inhibitors, anti-mitotic agents, or cell-cycle inhibitors
Compounds interacting with or binding tubulin
Compounds inhibiting mitotic kinesins or other motor proteins including but not limited to Eg5, CENP-E, MCAK, Kid, MKLP-1
Proteasome inhibitors
Heat shock protein inhibitors
Compounds targeting the anti-apoptotic function of Bcl-2, Bcl-x₁ and like molecules
Enzymes Hormones, hormone antagonists or hormone inhibitors, or inhibitors of steroid biosynthesis
Steroids
Cytokines, hypoxia-selective cytotoxins, inhibitors of cytokines, lymphokines, antibodies directed against cytokines or oral and parenteral tolerance induction strategies
Supportive agents
Antiinflammatory compounds such as but not limited to COX-2 inhibitors
Chemical radiation sensitizers and protectors
Photochemically activated drugs
Synthetic poly- or oligonucleotides
Other chemotherapeutic or naturally occurring, semi-synthetic or synthetic therapeutic agents, such as cytotoxic antibiotics, antibodies targeting surface molecules of cancer cells, antibodies targeting growth factors or their receptors, inhibitors of metalloproteinases, inhibitors of oncogenes, inhibitors of gene transcription or of RNA translation or protein expression, or complexes of rare earth elements.

In a preferred embodiment (8) with regard to any aspect of the invention the further chemotherapeutic agent 2 is selected from the group consisting of compounds interacting with or binding tubulin, synthetic small molecule VEGF receptor antagonists, small molecule growth factor receptor antagonists, inhibitors of the EGF receptor and/or HER2 receptor and/or VEGF receptor and/or integrin receptors or any other protein tyrosine kinase receptors which are not classified under the synthetic small-molecules, inhibitors directed to EGF receptor and/or HER2 receptor and/or VEGF receptor and/or VEGF and/or integrin receptors or any other protein tyrosine kinase receptors, which are fusion proteins, dihydropteridinone PLK-1 inhibitors such as disclosed in WO 2004/076454 (incorporated herewith by referencein uits entirety), compounds which interact with nucleic acids and which are classified as alkylating agents or platinum compounds, compounds which interact with nucleic acids and which are classified as anthracyclines, as DNA intercalators or as DNA cross-linking agents, including DNA minor-groove binding compounds, anti-metabolites, naturally occurring, semi-synthetic or synthetic bleomycin type antibiotics, inhibitors of DNA transcribing enzymes, and especially the topoisomerase I or topoisomerase II inhibitors, chromatin modifying agents, mitosis inhibitors, anti-mitotic agents, cell-cycle inhibitors, proteasome inhibitors, enzymes, hormones, hormone antagonists, hormone inhibitors, inhibitors of steroid biosynthesis, steroids, cytokines, hypoxia-selective cytotoxins, inhibitors of cytokines, lymphokines, antibodies directed against cytokines, oral and parenteral tolerance induction agents, supportive agents, chemical radiation sensitizers and protectors, photo-chemically activated drugs, synthetic poly- or oligonucleotides, optionally modified or conjugated, non-steroidal anti-inflammatory drugs, cytotoxic antibiotics, antibodies targeting the surface molecules of cancer cells, antibodies targeting growth factors or their receptors, inhibitors of metalloproteinases, metals, inhibitors of oncogenes, inhibitors of gene transcription or of RNA translation or protein expression, complexes of rare earth elements, and photo-chemotherapeutic agents.

Preferred embodiment (9) of the chemotherapeutic agent 2 include small molecule tyrosine kinase or serine/threonine kinase inhibitors, compounds interacting with nucleic acids classified as alkylating agents or anthracyclines, anti-metabolites, inhibitors of DNA transcribing enzymes such as topoisomerase I or II, tubulin binding drugs, anti-mitotic agents, antibodies targeting growth factors or their receptors, antibodies targeting VEGF or its receptors and antibodies binding to surface molecules of cancer cells or ligands of these surface molecules in form of the hydrates and/or solvates and optionally in the form of the individual optical isomers, mixtures of the individual enantiomers or racemates thereof.

In another preferred embodiment (10) of the invention the chemotherapeutic agent 2 is selected from the group consisting of a small molecule VEGF receptor antagonist such as vatalanib (PTK-787/ZK222584), SU-5416, SU-6668, SU-11248, SU-14813, AZD-6474, AZD-2171, CP-547632, CEP-7055, AG-013736, IM-842 or GW-786034, a dual EGFR/HER2 antagonist such as gefitinib, erlotinib, HKI-272, CI-1033 or GW-2016, an EGFR antagonist such as iressa (ZD-1839), tarceva (OSI-774), PKI-166, EKB-569 or herceptin, an antagonist of the mitogen-activated protein kinase such as BAY-43-9006 or BAY-57-9006, a protein kinase receptor antagonist which is not classified under the synthetic small molecules such as atrasentan, rituximab, cetuximab, Avastin™ (bevacizumab), bivatuzumab mertansine, IMC-1C11, erbitux (C-225), DC-101, EMD-72000, vitaxin, imatinib or dasatinib, a protein tyrosine kinase inhibitor which is a fusion protein such as VEGFtrap, an alkylating agent or a platinum compound such as melphalan, cyclophosphamide, an oxazaphosphorine, cisplatin, carboplatin, oxaliplatin, satraplatin, tetraplatin, iproplatin, mitomycin, streptozocin, carmustine (BCNU), lomustine (CCNU), busulfan, ifosfamide, streptozocin, thiotepa, chlorambucil, a nitrogen mustard such as mechlorethamine, an ethyleneimine compound, an alkylsulphonate, daunorubicin, doxorubicin (adriamycin), liposomal doxorubicin (doxil), epirubicin, idarubicin, mitoxantrone, amsacrine, dactinomycin, distamycin or a derivative thereof, netropsin, pibenzimol, mitomycin, CC-1065, a duocarmycin, mithramycin, chromomycin, olivomycin, a phtalanilide such as propamidine or stilbamidine, an anthramycin, an aziridine, a nitrosourea or a derivative thereof, a pyrimidine or purine analogue or antagonist or an inhibitor of the nucleoside diphosphate reductase such as cytarabine, 5-fluorouracile (5-FU), pemetrexed, tegafur/uracil, uracil mustard, fludarabine, gemcitabine, capecitabine, mercaptopurine, cladribine, thioguanine, methotrexate, pentostatin, hydroxyurea, or folic acid, a phleomycin, a bleomycin or a derivative or salt thereof, CHPP, BZPP, MTPP, BAPP, liblomycin, an acridine or a derivative thereof, a rifamycin, an actinomycin, adramycin, a camptothecin such as irinotecan (camptosar) or topotecan, an amsacrine or analogue thereof, a tricyclic carboxamide, an histonedeacetylase inhibitor such as SAHA, MD-275, trichostatin A, CBHA, LAQ824, or valproic acid, an anti-cancer drug from plants such as paclitaxel (taxol), docetaxel or taxotere, a vinca alkaloid such as navelbine, vinblastin, vincristin, vindesine or vinorelbine, a tropolone alkaloid such as colchicine or a derivative thereof, a macrolide such as maytansine, an ansamitocin or rhizoxin, an antimitotic peptide such as phomopsin or dolastatin, an epipodophyllotoxin or a derivative of podophyllotoxin such as etoposide or teniposide, a steganacin, an antimitotic carbamate derivative such as combretastatin or amphetinile, procarbazine, a proteasome inhibitor such as bortezomib, an enzyme such as asparaginase, pegylated asparaginase (pegaspargase) or a thymidine-phosphorylase inhibitor, a gestagen or an estrogen such as estramustine (T-66) or megestrol, an anti-androgen such as flutamide, casodex, anandron or cyproterone acetate, an aromatase inhibitor such as aminogluthetimide, anastrozole, formestan, exemestane or letrozole, a GNrH analogue such as leuprorelin, buserelin, goserelin or triptorelin, an anti-estrogen such as tamoxifen or its citrate salt, droloxifene, trioxifene, raloxifene or zindoxifene, an estrogen receptor antagonist such as fulvestrant, a derivative of 17β-estradiol such as ICI 164,384 or ICI 182,780, aminoglutethimide, formestane, fadrozole, finasteride, ketoconazole, a LH-RH antagonist such as leuprolide, a steroid such as prednisone, prednisolone, methylprednisolone, dexamethasone, budenoside, fluocortolone or triamcinolone, an interferon such as interferon β, an interleukin such as IL-10 or IL-12, an anti-TNFα antibody such as etanercept, TNF-α (tasonermin), an immunomodulatory drug such as thalidomide, its R- and S-enantiomers and its derivatives, or revimid (CC-5013), a leukotrien antagonist, mitomycin C, an aziridoquinone such as BMY-42355, AZQ or EO-9, a 2-nitroimidazole such as misonidazole, NLP-1 or NLA-1, a nitroacridine, a nitroquinoline, a nitropyrazoloacridine, a “dual-function” nitro aromatic such as RSU-1069 or RB-6145, CB-1954, a N-oxide of nitrogen mustard such as nitromin, a metal complex of a nitrogen mustard, an anti-CD3 or anti-CD25 antibody, a tolerance induction agent, a biphosphonate or derivative thereof such as minodronic acid or its derivatives (YM-529, Ono-5920, YH-529), zoledronic acid monohydrate, ibandronate sodium hydrate or clodronate disodium, a nitroimidazole such as metronidazole, misonidazole, benznidazole or nimorazole, a nitroaryl compound such as RSU-1069, a nitroxyl or N-oxide such as SR-4233, an halogenated pyrimidine analogue such as bromodeoxyuridine, iododeoxyuridine, a thiophosphate such as WR-2721, a photo-chemically activated drug such as porfimer, photofrin, a benzoporphyrin derivative, a pheophorbide derivative, merocyanin 540 (MC-540) or tin etioporpurin, an anti-template or an anti-sense RNA or DNA such as oblimersen, a non-steroidal inflammatory drug such as acetylsalicyclic acid, mesalazin, ibuprofen, naproxen, flurbiprofen, fenoprofen, fenbufen, ketoprofen, indoprofen, pirprofen, carprofen, oxaprozin, pranoprofen, miroprofen, tioxaprofen, suprofen, alminoprofen, tiaprofenic acid, fluprofen, indomethacin, sulindac, tolmetin, zomepirac, nabumetone, diclofenac, fenclofenac, alclofenac, bromfenac, ibufenac, aceclofenac, acemetacin, fentiazac, clidanac, etodolac, oxpinac, mefenamic acid, meclofenamic acid, flufenamic acid, nifluminic acid, tolfenamic acid, diflunisal, flufenisal, piroxicam, tenoxicam, lornoxicam, nimesulide, meloxicam, celecoxib, rofecoxib, or a pharmaceutically acceptable salt of a non-steroidal inflammatory drug, a cytotoxic antibiotic, an antibody targeting the surface molecules of cancer cells such as apolizumab or 1D09C3, an inhibitor of metalloproteinases such as TIMP-1 or TIMP-2, Zinc, an inhibitor of oncogenes such as P53 and Rb, a complex of rare earth elements such as the heterocyclic complexes of lanthanides, a photo-chemotherapeutic agent such as PUVA, an inhibitor of the transcription factor complex ESX/DRIP130/Sur-2, an inhibitor of HER-2 expression, such as the heat shock protein HSP90 modulator geldanamycin and its derivative 17-allylaminogeldanamycin or 17-AAG, or a therapeutic agent selected from IM-842, tetrathiomolybdate, squalamine, combrestatin A4, TNP-470, marimastat, neovastat, bicalutamide, abarelix, oregovomab, mitumomab, TLK-286, alemtuzumab, ibritumomab, temozolomide, denileukin diftitox, aldesleukin, dacarbazine, floxuridine, plicamycin, mitotane, pipobroman, plicamycin, tamoxifen and testolactone.

Preferred embodiment (11) of the chemotherapeutic agent 2 include small molecule VEGF receptor antagonist such as vatalanib (PTK-787/ZK222584), SU-5416, SU-6668, SU-11248, SU-14813, AZD-6474, EGFR/HER2 antagonists such as HKI-272, CI-1033 or GW-2016, an EGFR antagonist such as iressa (gefitinib, ZD-1839), tarceva (erlotinib, OSI-774), PKI-166, EKB-569 or herceptin, an antagonist of the mitogen-activated protein kinase such as BAY-43-9006 or BAY-57-9006, atrasentan, rituximab, cetuximab, Avastin™ (bevacizumab), IMC-1C11, erbitux (C-225), DC-101, EMD-72000, irinotecan, vitaxin, imatinib, an alkylating agent or a platinum compound such as melphalan, cyclophosphamide, cisplatin, carboplatin, oxaliplatin, satraplatin, daunorubicin, doxorubicin (adriamycin), liposomal doxorubicin (doxil), epirubicin, idarubicin, a pyrimidine or purine analogue or antagonist or an inhibitor of the nucleoside diphosphate reductase such as cytarabine, 5-fluorouracile (5-FU), pemetrexed, tegafur/uracil, gemcitabine, capecitabine, mercaptopurine, methotrexate, an anti-cancer drug such as paclitaxel (taxol) or docetaxel, a vinca alkaloid such as navelbine, vinblastin, vincristin, vindesine or vinorelbine, an antimitotic peptide such as dolastatin, an epipodophyllotoxin or a derivative of podophyllotoxin such as etoposide or teniposide, a non-steroidal inflammatory drug such as meloxicam, celecoxib, rofecoxib, an antibody targeting the surface molecules of cancer cells such as apolizumab or 1D09C3 or the heat shock protein HSP90 modulator geldanamycin and its derivative 17-allylaminogeldanamycin or 17-AAG.

In another preferred embodiment (12) of the instant invention the chemotherapeutic agent 2 is selected from the group consisting of an anti-cancer drug from plants such as irinotecan, paclitaxel (taxol), docetaxel, a vinca alkaloid such as navelbine, vinblastin, vincristin, vindesine or vinorelbine, an alkylating agent or a platinum compound such as melphalan, cyclophosphamide, an oxazaphosphorine, cisplatin, carboplatin, oxaliplatin, satraplatin, tetraplatin, iproplatin, mitomycin, streptozocin, carmustine (BCNU), lomustine (CCNU), busulfan, ifosfamide, streptozocin, thiotepa, chlorambucil, a nitrogen mustard such as mechlorethamine, an immunomodulatory drug such as thalidomide, its R- and S-enantiomers and its derivatives, or revimid (CC-5013)), an ethyleneimine compound, an alkylsulphonate, daunorubicin, doxorubicin (adriamycin), liposomal doxorubicin (doxil), epirubicin, idarubicin, mitoxantrone, amsacrine, dactinomycin, distamycin or a derivative thereof, netropsin, pibenzimol, mitomycin, CC-1065, a duocarmycin, mithramycin, chromomycin, olivomycin, a phtalanilide such as propamidine or stilbamidine, an anthramycin, an aziridine, a nitrosourea or a derivative thereof, a pyrimidine or purine analogue or antagonist or an inhibitor of the nucleoside diphosphate reductase such as cytarabine, 5-fluorouracile (5-FU), uracil mustard, fludarabine, gemcitabine, capecitabine, mercaptopurine, cladribine, thioguanine, methotrexate, pentostatin, hydroxyurea, or folic acid, an acridine or a derivative thereof, a rifamycin, an actinomycin, adramycin, a camptothecin such as irinotecan (camptosar) or topotecan, an amsacrine or analogue thereof, a tricyclic carboxamide, an histonedeacetylase inhibitor such as SAHA, MD-275, trichostatin A, CBHA, LAQ824, or valproic acid, a proteasome inhibitor such as bortezomib, a small molecule VEGF receptor antagonist such as vatalanib (PTK-787/ZK222584), SU-5416, SU-6668, SU-11248, SU-14813, AZD-6474, AZD-2171, CP-547632, CEP-7055, AG-013736, IM-842 or GW-786034, an antagonist of the mitogen-activated protein kinase such as BAY-43-9006 or BAY-57-9006, a dual EGFR/HER2 antagonist such as HKI-272, CI-1033 or GW-2016, an EGFR antagonist such as iressa (ZD-1839), tarceva (OSI-774), PKI-166, EKB-569 or herceptin, an inhibitor of the transcription factor complex ESX/DRIP130/Sur-2, an inhibitor of HER-2 expression, such as the heat shock protein HSP90 modulator geldanamycin and its derivative 17-allylaminogeldanamycin or 17-AAG, a protein kinase receptor antagonist which is not classified under the synthetic small molecules such as atrasentan, rituximab, cetuximab, Avastin™ (bevacizumab), bivatuzumab mertansine, IMC-1C11, erbitux (C-225), DC-101, EMD-72000, vitaxin, imatinib, and an antibody targeting the surface molecules of cancer cells such as apolizumab or 1D09C3.

Preferred embodiment (13) of the chemotherapeutic agent 2 include small molecule receptor antagonists such as vatalanib, SU 11248 or AZD-6474, EGFR, HER2 or EGFR/HER2 antagonists such as gefitinib, erlotinib, HKI-272, CI-1033 or Herceptin, antibodies such as bevacizumab, cetuximab or rituximab, DNA alkylating drugs such as cisplatin, oxaliplatin or carboplatin, anthracyclines such as doxorubicin or epirubicin, an antimetabolite such as 5-FU, pemetrexed, gemcitabine or capecitabine, a camptothecin such as irinotecan or topotecan, an anti-cancer drug such as paclitaxel or docetaxel, an epipodophyllotoxin such as etoposide or teniposide, a proteasome inhibitor such as bortezomib or antiinflammatory drugs such as celecoxib or rofecoxib, optionally in form of the pharmaceutically acceptable salts, in form of the hydrates and/or solvates and optionally in the form of the individual optical isomers, mixtures of the individual enantiomers or racemates thereof.

In another preferred embodiment (14) of the instant invention the chemotherapeutic agent 2 is 3-Z-[1-(4-(N-((4-methyl-piperazin-1-yl)-methylcarbonyl)-N-methyl-amino)-anilino)-1-phenyl-methylene]-6-methoxycarbonyl-2-indolinone, or a polymorph, metabolite or pharmaceutically acceptable salt thereof.

In another preferred embodiment (15) of the instant invention the chemotherapeutic agent 2 is the monoethanesulfonate salt of 3-Z-[1-(4-(N-((4-methyl-piperazin-1-yl)-methylcarbonyl)-N-methyl-amino)-anilino)-1-phenyl-methylene]-6-methoxycarbonyl-2-indolinone.

In another preferred embodiment (16) of the instant invention the chemotherapeutic agent 2 is 3-Z-[1-(4-dimethylaminomethylanilino)-1-(4-(2-carboxyethyl)phenyl)methylene]-6-fluoro-2-indolinone.

In another preferred embodiment (17) of the instant invention the chemotherapeutic agent 2 is 4-[[(7R)-8-(cyclopentyl)-7-ethyl-5,6,7,8-tetrahydro-5-methyl-6-oxo-2-pteridinyl]amino]-N-3-methoxy-N—(N-methyl-4-piperidinyl)-benzamide,

In another preferred embodiment (18) of the instant invention the chemotherapeutic agent 2 is N-[trans-4-[4-(cyclopropylmethyl)-1-piperazinyl]cyclohexyl]-4-[[(7R)-7-ethyl-5,6,7,8-tetrahydro-5-methyl-8-(1-methylethyl)-6-oxo-2-pteridinyl]amino]-3-methoxy-benzamide.

In another preferred embodiment (19) of the instant invention the chemotherapeutic agent 2 is irinotecan, 5 FU, leucovorine, topotecan, oxaliplatin, docetaxel, paclitaxel, gemcitabine, pemetrexed, cisplatin, carboplatin, bevacizumab, cetuximab, gefitinib or erlotinib, particularly preferred irinotecan, 5 FU, leucovorine, docetaxel, gemcitabine, topotecan or paclitaxel.

In another preferred embodiment (20) of the instant invention the chemotherapeutic agent 2 is a compound which reduces the transport of hyaluronan mediated by one or more ABC transporters, or drug transport inhibitor, such as a P-glycoprotein (P-gp) inhibitor molecule or inhibitor peptide, an MRP1 inhibitor, an antibody directed against and capable of blocking the ABC transporter, an antisense oligomer, iRNA, siRNA or aptamer directed against one or more ABC transporters. Examples of P-glycoprotein (P-gp) inhibitor molecules in accordance with the present invention are zosuquidar (LY 335973), its salts (especially the trichloride salt) and its polymorphs, cyclosporin A (also known as cyclosporine), verapamil or its R-isomer, tamoxifen, quinidine, d-alpha tocopheryl polyethylene glycol 1000 succinate, VX-710, PSC833, phenothiazine, GF120918 (II), SDZ PSC 833, TMBY, MS-073, S-9788, SDZ 280-446, XR(9051) and functional derivatives, analogues and isomers of these.

Furthermore, where any of the compounds 2 carries an acidic moiety, suitable pharmaceutically acceptable salts thereof may include alkali metal salts (e.g. sodium or potassium salts), alkaline earth metal salts (e. g. calcium or magnesium salts) and salts formed with suitable organic ligands (e.g. quaternary ammonium salts).

The compounds 2 may have chiral centers and may occur as racemates, racemic mixtures and as individual diastereomers, or enantiomers with all isomeric forms being included in the present invention. Hence, where a compound is chiral, the separate enantiomers, substantially free of the others, are included within the scope of the invention. Further included are all mixtures of the two enantiomers. Also included within the scope of the invention are polymorphs and hydrates of the compounds of the instant invention.

The present invention includes within its scope prodrugs of a compound 1 of formula (I) and of the further active ingredient 2. In general, such prodrugs will be functional derivatives of the compounds or active ingredients of this invention which are readily convertible in vivo into the required compound.

In a further embodiment the invention relates to a composition as defined hereinbefore, which inhibits the proliferation of various human tumour cell lines including but not limited to MDA-MB-435S, MDA-MB453, HT29, FaDu, SKOV-3, DU145, PC-3, NCI-N87, and A431,

In the context of the instant invention the indication “cancer” preferably is selected from the group (21) consisting of solid tumours, e.g. from the group consisting of carcinomas, sarcomas, melanomas, myelomas, hematological neoplasias, lymphomas and childhood cancers.

Examples of carcinomas within the scope of the invention include but are not limited to the group (22) consisting of adenocarcinoma (AC), squamous cell carcinoma (SCC) and mixed or undifferentiated carcinomas. Carcinomas within the scope of the invention include but are not limited to the following histologies:
Head and neck tumours: SCC, AC, transitional cell cancers, mucoepidermoid cancers, undifferentiated carcinomas;
Central nervous system tumours: Astrocytoma, glioblastoma, meningeoma, neurinoma, schwannoma, ependymoma, hypophysoma, oligodendroglioma, medulloblastoma;
Bronchial and mediastinal tumours:
   Bronchial tumours:
      Small cell lung cancers (SCLC): oat-cell lung cancer, intermediate cell cancer, combined oat-cell lung cancer;
      Non-small cell lung cancers (NSCLC): SCC, spindle cell carcinoma, AC, bronchioalveolar carcinoma, large cell NSCLC, clear cell NSCLC;
   Mesothelioma;
   Thymoma;
   Thyroid carcinomas: papillary, follicular, anaplastic, medullary;
Tumours of the gastrointestinal tract:
   Oesophageal cancers: SCC, AC, anaplastic, carcinoid, sarcoma;
   Gastric cancers: AC, adenosquamous, anaplastic;
   Colorectal cancers: AC, including hereditary forms of AC, carcinoid, sarcoma;
   Anal cancers: SCC, transitional epithelial cancer, AC, basal cell carcinoma;
   Pancreatic cancers: AC, including ductal and acinary cancers, papillary, adenosquamous, undifferentiated, tumours of the endocrine pancreas;
   Hepatocellular carcinoma, cholangiocarcinoma, angiosarcoma, hepatoblastoma;
   Biliary carcinomas: AC, SCC, small cell, undifferentiated;
   Gastrointestinal stroma tumours (GIST);
Gynaecological cancers:
   Breast cancers: AC, including invasive ductal, lobular and medullary cancers, tubular, mucinous cancers, Paget-carcinoma, inflammatory carcinoma, ductal and lobular carcinoma in situ;
   Ovarian cancers: Epithelial tumours, stroma tumours, germ cell tumours, undifferentiated tumours;
   Cervical cancers: SCC, AC, mixed and undifferentiated tumours;
   Endometrial cancers: AC, SCC, mixed, undifferentiated tumours;
   Vulvar cancers: SCC, AC;
   Vaginal cancers: SCC, AC;
Urinary tract and testicular cancers:
   Testicular cancers: seminoma;
   Non-seminomatous germ cell tumours: teratoma, embryonal cell carcinoma, choriocarcinoma, yolk sac tumour, mixed, Sertoli and Leydig-cell tumours;
   Extragonadal germ cell tumours;
   Prostate cancers: AC, small cell, SCC;
   Renal cell cancers: AC, including clear cell, papillary and chromophobous carcinomas, hereditary forms (e.g. von-Hippel-Lindau syndrome), nephroblastoma;
   Urinary bladder cancers: transitional cell (urothelial) cancers, SCC, AC;
   Urethral cancers: SCC, transitional cell cancers, AC;
   Penile cancers: SCC;
Tumours of endocrine tissue:
   Thyroid cancers: papillary, follicular, anaplastic, medullary carcinomas, including MEN syndrome;
   Tumours of the endocrine pancreas;
   Carcinoids;
   Pheochromocytoma.

Examples (23) of sarcomas within the scope of the invention include but are not limited to Ewing-sarcoma, osteosarcoma or osteogenic sarcoma, chondrosarcoma, synovial sarcoma, leiomyosarcoma, rhabdomyosarcoma, mesothelial sarcoma or mesothelioma, fibrosarcoma, angiosarcoma or hemangioendothelioma, liposarcoma, glioma or astrocytoma, myxosarcoma, malignant fibrous histiocytoma, mesenchymous or mixed mesodermal tumour, neuroblastoma and clear cell sarcoma.

Examples (24) of melanomas within the scope of the invention include but are not limited to superficial spreading melanoma, nodular and lentigo-maligna melanoma.

Examples (25) of myelomas within the scope of the invention include but are not limited to immunocytoma, plasmocytoma and multiple myeloma.

In another preferred embodiment (26) the invention relates to the use according to the invention, wherein the hematological neoplasia is leukemia.

Further examples (27) of hematologic neoplasias within the scope of the invention include but are not limited to acute or chronic leukemias of myeloid, erythroid or lymphatic origin, myelodysplastic syndromes (MDS) and myeloproliferative syndromes (MPS, such as chronic myelogeneous leukemia, osteomyelofibrosis, polycythemia vera or essential thrombocythemia).

Examples of lymphomas (28) within the scope of the invention include but are not limited to:
Hodgkin's-lymphoma;
Non-Hodgkin's-lymphomas: T- and B-cell lymphomas
   B-cell lymphomas:
      Low and intermediate grade: Chronic lymphocytic leukemia (CLL), prolymphocytic leukemia (PLL), small lymphocytic lymphoma, hairy cell leukemia, plasmacytoid lymphoma, mantle cell lymphoma, follicular lymphoma, marginal zone lymphoma including MALT-lymphoma;
      High grade: diffuse large B-cell lymphoma (DLBCL including immunoblastic and centroblastic variants), lymphoblastic, Burkitt's lymphoma;
   T-cell lymphomas:
      Low grade: T-CLL, T-PLL, Mycosis fungoides, Sezary-syndrome;
      High grade: Anaplastic large cell, T-immunoblastic and lymphoblastic.

In another preferred embodiment (29) the invention relates to the use according to the invention, wherein the disease is cancer selected from the group consisting of mixed tumours, undifferentiated tumours and metastases thereof.

Examples (30) of mixed tumours within the scope of the invention include but are not limited to adenosquamous carcinomas, mixed mesodermal tumours, carcinosarcomas and teratocarcinomas.

Examples (31) of undifferentiated, other tumours or metastases thereof within the scope of the invention include but are not limited to undifferentiated tumours, carcinomas of unknown primary (CUP), metastases of unknown primary (MUP) and pheochromocytoma, carcinoids.

Additionally the following tumour diseases (32) which can be treated with a compound of formula (I) in accordance with the invention are summarized:

acral lentiginous melanoma, actinic keratoses, adenoid cycstic carcinoma, adenomas, adenosarcoma, adrenocortical carcinoma, AIDS-related lymphoma, bartholin gland carcinoma, brain stem glioma, capillary carcinoma, central nervous system lymphoma, chondosarcoma, choriod plexus papilloma/carcinoma, cystadenoma, endodermal sinus tumor, endometrial hyperplasia, endometrial stromal sarcoma, endometrioid adenocarcinoma, epitheloid, focal nodular hyperplasia, gastrinoma, gestational trophoblastic tumor, glucagonoma, hepatic adenoma, hepatic adenomatosis, hypopharyngeal cancer, hypothalamic and visual pathway glioma, insulinoma, intraepithelial neoplasia, interepithelial squamous cell neoplasia, intraocular invasive squamous cell carcinoma, large cell carcinoma, islet cell carcinoma, Kaposi's sarcoma, laryngeal cancer, leukemia-related disorders, lip and oral cavity cancer, malignant mesothelial tumors, malignant thymoma, medulloepithelioma, merkel cell carcinoma, mucoepidermoid carcinoma, multiple myeloma/plasma cell neoplasm, mycosis fungoides, myelodysplastic syndrome, myeloproliferative disorders, nasal cavity and paranasal sinus cancer, nasopharyngeal cancer, neuroepithelial adenocarcinoma, nodular melanoma, oat cell carcinoma, oligodendroglial, oral cancer, oropharyngeal cancer, pineal cell, pituitary tumors, pseudosarcoma, pulmonary blastoma, parathyroid cancer, pineal and supratentorial primitive neuroectodermal tumors, pituitary tumor, plasma cell neoplasm, pleuropulmonary blastoma, retinoblastoma, serous carcinoma, small intestine cancer, soft tissue carcinomas, somatostatin-secreting tumor, supratentorial primitive neuroectodermal tumors, uveal melanoma, verrucous carcinoma, vipoma, Waldenstrom's macroglobulinemia, well differentiated carcinoma, and Wilm's tumor.

In the context of the instant invention any hormone sensitive cancer indication which can be influenced by hormones, such as prostate cancer, breast cancers, and carcinoid syndrome, can be treated using a combination of two chemotherapeutic agents 2, one of them being a enzyme hormones, hormone antagonist, hormone inhibitor, steroid or an inhibitors of steroid biosynthesis.

In a preferred embodiment A, with regard to the first, second and third aspect of the invention, compound 1 of formula (I) is selected from the group consisting of
(a) 4-[(3-chloro-4-fluorophenyl)amino]-6-{[4-(N,N-dimethylamino)-1-oxo-2-buten-1-yl]amino}-7-cyclobutyloxy-quinazoline,
(b) 4-[(3-chloro-4-fluorophenyl)amino]-6-{[4-(N,N-dimethylamino)-1-oxo-2-buten-1-yl]amino}-7-cyclopentyloxy-quinazoline,
(c) 4-[(3-chloro-4-fluorophenyl)amino]-6-{[4-(N,N-dimethylamino)-1-oxo-2-buten-1-yl]amino}-7-((R)-tetrahydrofuran-3-yloxy)-quinazoline,
(d) 4-[(3-chloro-4-fluorophenyl)amino]-6-{[4-(N,N-dimethylamino)-1-oxo-2-buten-1-yl]amino}-7-((S)-tetrahydrofuran-3-yloxy)-quinazoline (BIBW2992),
(e) 4-[(3-chloro-4-fluorophenyl)amino]-6-{[4-(N,N-dimethylamino)-1-oxo-2-buten-1-yl]amino}-7-(tetrahydropyran-4-yloxy)-quinazoline,
(f) 4-[(3-chloro-4-fluorophenyl)amino]-6-{[4-(N,N-dimethylamino)-1-oxo-2-buten-1-yl]amino}-7-[(tetrahydrofuran-2-yl)methoxy]-quinazoline,
(g) 4-[(3-chloro-4-fluorophenyl)amino]-6-{[4-(N,N-dimethylamino)-1-oxo-2-buten-1-yl]amino}-7-[(tetrahydrofuran-3-yl)methoxy]-quinazoline,
(h) 4-[(R)-(1-phenyl-ethyl)amino]-6-{[4-(N,N-dimethylamino)-1-oxo-2-buten-1-yl]amino}-7-cyclopropylmethoxy-quinazoline,
(i) 4-[(3-chloro-4-fluorophenyl)amino]-6-{[4-(morpholin-4-yl)-1-oxo-2-buten-1-yl]amino}-7-[(tetrahydrofuran-2-yl)methoxy]-quinazoline,
(j) 4-[(3-chloro-4-fluorophenyl)amino]-6-{[4-(N,N-dimethylamino)-1-oxo-2-buten-1-yl]amino}-7-[(S)-(tetrahydrofuran-2-yl)methoxy]-quinazoline,
(k) 4-[(3-chloro-4-fluoro-phenyl)amino]-6-{[4-(homomorpholin-4-yl)-1-oxo-2-buten-1-yl]amino}-7-[(S)-(tetrahydrofuran-3-yl)oxy]-quinazoline, and
(r) 4-[(3-chloro-4-fluoro-phenyl)amino]-6-{[4-(dimethylamino)-1-oxo-2-buten-1-yl]amino}-7-cyclopropylmethoxy-quinazoline,
   the chemotherapeutic agent 2 is selected from the group consisting of
   vatalanib (PTK-787/ZK222584), SU-5416, SU-6668, SU-11248, SU-14813, AZD-6474, AZD-2171, CP-547632, CEP-7055, AG-013736, IM-842 or GW-786034, gefitinib, erlotinib, CI-1033 or GW-2016, iressa (ZD-1839), tarceva (OSI-774), PKI-166, EKB-569, HKI-272, herceptin, BAY-43-9006, BAY-57-9006, atrasentan, rituximab, cetuximab, Avastin™ (bevacizumab), IMC-1C11, erbitux (C-225), DC-101, EMD-72000, vitaxin, imatinib, dasatinib, VEGFtrap, melphalan, an oxazaphosphorine, carboplatin, oxaliplatin, satraplatin, tetraplatin, iproplatin, mitomycin, streptozocin, carmustine (BCNU), lomustine (CCNU), busulfan, ifosfamide, streptozocin, thiotepa, chlorambucil, mechlorethamine, daunorubicin, liposomal doxorubicin (doxil), epirubicin, idarubicin, mitoxantrone, amsacrine, dactinomycin, distamycin or a derivative thereof, netropsin, pibenzimol, mitomycin, CC-1065, a duocarmycin, mithramycin, chromomycin, olivomycin, propamidine or stilbamidine, an anthramycin, an aziridine, cytarabine, pemetrexed, tegafur/uracil, uracil mustard, fludarabine, gemcitabine, capecitabine, mercaptopurine, cladribine, thioguanine, methotrexate, pentostatin, hydroxyurea, or folic acid, a phleomycin, a bleomycin or a derivative or salt thereof, CHPP, BZPP, MTPP, BAPP, liblomycin, an acridine or a derivative thereof, a rifamycin, an actinomycin, adramycin, irinotecan (camptosar), topotecan, SAHA, MD-275, trichostatin A, CBHA, LAQ824, valproic acid, paclitaxel (taxol), docetaxel, taxotere, navelbine, vinblastin, vincristin, vindesine, vinorelbine, colchicine or a derivative thereof, maytansine, phomopsin, dolastatin, teniposide, a steganacin, combretastatin, amphetinile, procarbazine, bortezomib, asparaginase, pegylated asparaginase (pegaspargase), estramustine (T-66), megestrol, flutamide, casodex, anandron, cyproterone acetate, aminogluthetimide, anastrozole, formestan, exemestane or letrozole, leuprorelin, buserelin, goserelin, triptorelin, droloxifene, trioxifene, raloxifene, zindoxifene, fulvestrant, ICI 164,384, ICI 182,780, aminoglutethimide, formestane, fadrozole, finasteride, ketoconazole, leuprolide, prednisone, prednisolone, methylprednisolone, dexamethasone, budenoside, fluocortolone, triamcinolone, interferon β, IL-10, IL-12, etanercept, thalidomide, its R- and S-enantiomers and its derivatives, revimid (CC-5013), mitomycin C, BMY-42355, AZQ, EO-9, NLP-1, NLA-1, a nitroacridine, RSU-1069, RB-6145, CB-1954, nitromin, minodronic acid and its derivatives (YM-529, Ono-5920, YH-529), zoledronic acid monohydrate, ibandronate sodium hydrate, clodronate disodium, metronidazole, misonidazole, benznidazole, nimorazole, RSU-1069, SR-4233, bromodeoxyuridine, iododeoxyuridine, WR-2721, porfimer, photofrin, merocyanin 540 (MC-540), tin etioporpurin, oblimersen, acetylsalicyclic acid, mesalazin, ibuprofen, naproxen, flurbiprofen, fenoprofen, fenbufen, ketoprofen, indoprofen, pirprofen, carprofen, oxaprozin, pranoprofen, miroprofen, tioxaprofen, suprofen, alminoprofen, tiaprofenic acid, fluprofen, indomethacin, sulindac, tolmetin, zomepirac, nabumetone, diclofenac, fenclofenac, alclofenac, bromfenac, ibufenac, aceclofenac, acemetacin, fentiazac, clidanac, etodolac, oxpinac, mefenamic acid, meclofenamic acid, flufenamic acid, nifluminic acid, tolfenamic acid, diflunisal, flufenisal, piroxicam, tenoxicam, lornoxicam, nimesulide, meloxicam, celecoxib, rofecoxib, apolizumab, 1D09C3, TIMP-1, TIMP-2, Zinc, P53, Rb, PUVA, the heat shock protein HSP90 modulator geldanamycin, 17-allylaminogeldanamycin, 17-AAG, IM-842, tetrathiomolybdate, squalamine, combrestatin A4, TNP-470, marimastat, neovastat, bicalutamide, abarelix, oregovomab, mitumomab, TLK-286, alemtuzumab, ibritumomab, bivatuzumab mertansine, temozolomide, denileukin diftitox, aldesleukin, dacarbazine, floxuridine, plicamycin, mitotane, pipobroman, plicamycin, tamoxifen and testolacton,
4-[[(7R)-8-(cyclopentyl)-7-ethyl-5,6,7,8-tetrahydro-5-methyl-6-oxo-2-pteridinyl]-amino]-N-3-methoxy-N—(N-methyl-4-piperidinyl)-benzamide; and
N-[trans-4-[4-(cyclopropylmethyl)-1-piperazinyl]cyclohexyl]-4-[[(7R)-7-ethyl-5,6,7,8-tetrahydro-5-methyl-8-(1-methylethyl)-6-oxo-2-pteridinyl]amino]-3-methoxy-benzamide;
   or the chemotherapeutic agent 2 is selected from the group consisting of
   cyclophosphamide, cisplatin, doxorubicin (adriamycin), 5-fluorouracile (5-FU), etoposide and tamoxifen or its citrate salt,
   or in a particular preferred subgenus the chemotherapeutic agent 2 is selected from the group consisting of

BAY-43-9006, BAY-57-9006, atrasentan, rituximab, cetuximab, Avastin™ (bevacizumab), IMC-1C11, erbitux (C-225), DC-101, EMD-72000, vitaxin, imatinib, melphalan, carboplatin, oxaliplatin, satraplatin, daunorubicin, liposomal doxorubicin (doxil), epirubicin, idarubicin, cytarabine, pemetrexed, tegafur/uracil, gemcitabine, capecitabine, mercaptopurine, methotrexate, paclitaxel (taxol), docetaxel, navelbine, vincristin, vindesine, vinorelbine, dolastatin, teniposide, meloxicam, celecoxib, rofecoxib, apolizumab, 1D09C3, the heat shock protein HSP90 modulator geldanamycin, 17-allylaminogeldanamycin, 17-AAG,
4-[[(7R)-8-(cyclopentyl)-7-ethyl-5,6,7,8-tetrahydro-5-methyl-6-oxo-2-pteridinyl]-amino]-N-3-methoxy-N—(N-methyl-4-piperidinyl)-benzamide; and
N-[trans-4-[4-(cyclopropylmethyl)-1-piperazinyl]cyclohexyl]-4-[[(7R)-7-ethyl-5,6,7,8-tetrahydro-5-methyl-8-(1-methylethyl)-6-oxo-2-pteridinyl]amino]-3-methoxy-benzamide;
   and the cancer indication is selected from the group consisting of
   Head and neck tumours: SCC, AC, transitional cell cancers, mucoepidermoid cancers, undifferentiated carcinomas;
   Central nervous system tumours: Astrocytoma, glioblastoma, meningeoma, neurinoma, schwannoma, ependymoma, hypophysoma, oligodendroglioma, medulloblastoma;
   Bronchial and mediastinal tumours:
      Bronchial tumours:
         Non-small cell lung cancers (NSCLC): SCC, spindle cell carcinoma, AC, bronchioalveolar carcinoma, large cell NSCLC, clear cell NSCLC;
      Thyroid carcinomas: papillary, follicular, anaplastic, medullary;
   Tumours of the gastrointestinal tract:
      Oesophageal cancers: SCC, AC, anaplastic;
      Gastric cancers: AC, adenosquamous, anaplastic;
      Colorectal cancers: AC, including hereditary forms of AC, carcinoid, sarcoma;
      Pancreatic cancers: AC, including ductal and acinary cancers, papillary, adenosquamous, undifferentiated, tumours of the endocrine pancreas;
      Hepatocellular cancers, cholangiocarcinoma
   Gynaecological cancers:
      Breast cancers: AC, including invasive ductal, lobular and medullary cancers, tubular, mucinous cancers, Paget-carcinoma, inflammatory carcinoma, ductal and lobular carcinoma in situ;
      Ovarian cancers: Epithelial tumours, stroma tumours, germ cell tumours, undifferentiated tumours;
   Urinary tract and testicular cancers:
      Prostate cancers: AC, small cell, SCC;
      Renal cell cancers: AC, including clear cell, papillary and chromophobous carcinomas, hereditary forms (e.g. von-Hippel-Lindau syndrome), Wilm's tumor, nephroblastoma;
      Urinary bladder cancers: transitional cell (urothelial) cancers, SCC, AC.

Examples of sarcomas within the scope of the invention include but are not limited to Ewing-sarcoma, osteosarcoma or osteogenic sarcoma, chondrosarcoma, synovial sarcoma, leiomyosarcoma, rhabdomyosarcoma, mesothelial sarcoma or mesothelioma, fibrosarcoma, angiosarcoma or hemangioendothelioma, liposarcoma, glioma or astrocytoma, myxosarcoma, malignant fibrous histiocytoma, mesenchymous or mixed mesodermal tumour, neuroblastoma and clear cell sarcoma.

In a preferred embodiment B, with regard to the first, second and third aspect of the invention, compound 1 of formula (I) is selected from the group consisting of
(d) 4-[(3-chloro-4-fluorophenyl)amino]-6-{[4-(N,N-dimethylamino)-1-oxo-2-buten-1-yl]amino}-7-((S)-tetrahydrofuran-3-yloxy)-quinazoline (BIBW2992),
(k) 4-[(3-chloro-4-fluoro-phenyl)amino]-6-{[4-(homomorpholin-4-yl)-1-oxo-2-buten-1-yl]amino}-7-[(S)-(tetrahydrofuran-3-yl)oxy]-quinazoline,
   the chemotherapeutic agent 2 is selected from the group consisting of

BAY-43-9006, BAY-57-9006, atrasentan, rituximab, cetuximab, Avastin™ (bevacizumab), IMC-1C11, erbitux (C-225), DC-101, EMD-72000, vitaxin, imatinib, melphalan, carboplatin, oxaliplatin, satraplatin, daunorubicin, liposomal doxorubicin (doxil), epirubicin, idarubicin, cytarabine, pemetrexed, tegafur/uracil, gemcitabine, capecitabine, mercaptopurine, methotrexate, paclitaxel (taxol), docetaxel, navelbine, vincristin, vindesine, vinorelbine, dolastatin, teniposide, meloxicam, celecoxib, rofecoxib, apolizumab, 1D09C3, the heat shock protein HSP90 modulator geldanamycin, 17-allylaminogeldanamycin, 17-AAG,
4-[[(7R)-8-(cyclopentyl)-7-ethyl-5,6,7,8-tetrahydro-5-methyl-6-oxo-2-pteridinyl]-amino]-N-3-methoxy-N—(N-methyl-4-piperidinyl)-benzamide; and
N-[trans-4-[4-(cyclopropylmethyl)-1-piperazinyl]cyclohexyl]-4-[[(7R)-7-ethyl-5,6,7,8-tetrahydro-5-methyl-8-(1-methylethyl)-6-oxo-2-pteridinyl]amino]-3-methoxy-benzamide;
   or the chemotherapeutic agent 2 is selected from the group consisting of
   cyclophosphamide, cisplatin, doxorubicin (adriamycin), 5-fluorouracile (5-FU), etoposide and tamoxifen or its citrate salt,
   and the cancer indication is selected from the group consisting of
   Head and neck tumours: SCC, AC, transitional cell cancers, mucoepidermoid cancers, undifferentiated carcinomas;
   Colorectal cancers, metastatic or non-metastatic: AC, including hereditary forms of AC, carcinoid, sarcoma;
   Pancreatic cancers: AC, including ductal and acinary cancers, papillary, adenosquamous, undifferentiated, tumours of the endocrine pancreas;
   Breast cancers, metastatic or non-metastatic: AC, including invasive ductal, lobular and medullary cancers, tubular, mucinous cancers, Paget-carcinoma, inflammatory carcinoma, ductal and lobular carcinoma in situ;
   Prostate cancers: AC, small cell, SCC;
   Gastric cancers: AC, adenosquamous, anaplastic;
   Ovarian cancer;
   Non-small cell lung cancers (NSCLC): SCC, spindle cell carcinoma, AC, bronchioalveolar carcinoma, large cell NSCLC, clear cell NSCLC.

In a preferred embodiment C, with regard to the first, second and third aspect of the invention, compound 1 of formula (I) is selected from the group consisting of
(d) 4-[(3-chloro-4-fluorophenyl)amino]-6-{[4-(N,N-dimethylamino)-1-oxo-2-buten-1-yl]amino}-7-((S)-tetrahydrofuran-3-yloxy)-quinazoline (BIBW2992), and
(k) 4-[(3-chloro-4-fluoro-phenyl)amino]-6-{[4-(homomorpholin-4-yl)-1-oxo-2-buten-1-yl]amino}-7-[(S)-(tetrahydrofuran-3-yl)oxy]-quinazoline,
   the chemotherapeutic agent 2 is selected from the group consisting of
   vatalanib, SU 11248, AZD-6474, gefitinib, erlotinib, CI-1033, Herceptin, bevacizumab, cetuximab, rituximab, oxaliplatin, carboplatin, epirubicin, pemetrexed, gemcitabine,
   capecitabine, irinotecan, topotecan, paclitaxel, docetaxel, teniposide, bortezomib, celecoxib, rofecoxib,
   or the chemotherapeutic agent 2 is selected from the group consisting of
   cisplatin, doxorubicin (adriamycin), 5-fluorouracile (5-FU) and etoposide,
   and the cancer indication is selected from the group consisting of
   Head and neck tumours: SCC, AC, transitional cell cancers, mucoepidermoid cancers, undifferentiated carcinomas;
   Colorectal cancers, metastatic or non-metastatic: AC, including hereditary forms of AC, carcinoid, sarcoma;
   Pancreatic cancers: AC, including ductal and acinary cancers, papillary, adenosquamous, undifferentiated, tumours of the endocrine pancreas;
   Breast cancers, metastatic or non-metastatic: AC, including invasive ductal, lobular and medullary cancers, tubular, mucinous cancers, Paget-carcinoma, inflammatory carcinoma, ductal and lobular carcinoma in situ;
   Prostate cancers: AC, small cell, SCC;
   Non-small cell lung cancers (NSCLC): SCC, spindle cell carcinoma, AC, bronchioalveolar carcinoma, large cell NSCLC, clear cell NSCLC.

In a preferred embodiment D, with regard to the first, second and third aspect of the invention, compound 1 of formula (I) is selected from the group consisting of
(d) 4-[(3-chloro-4-fluorophenyl)amino]-6-{[4-(N,N-dimethylamino)-1-oxo-2-buten-1-yl]amino}-7-((S)-tetrahydrofuran-3-yloxy)-quinazoline (BIBW2992) or a pharmacologically acceptable salt thereof, preferably the dimaleate salt (d′), and
(k) 4-[(3-chloro-4-fluoro-phenyl)amino]-6-{[4-(homomorpholin-4-yl)-1-oxo-2-buten-1-yl]amino}-7-[(S)-(tetrahydrofuran-3-yl)oxy]-quinazoline,
   the chemotherapeutic agent 2 is selected from the group consisting of
   irinotecan, topotecan, oxaliplatin, docetaxel, paclitaxel, gemcitabine, pemetrexed, carboplatin, bevacizumab, cetuximab, gefitinib, erlotinib and estramustine,
   or the chemotherapeutic agent 2 is selected from the group consisting of
   cisplatin and 5-fluorouracile (5-FU),
   and the cancer indication is selected from the group consisting of
   Head and neck tumours: SCC, AC, transitional cell cancers, mucoepidermoid cancers, undifferentiated carcinomas;
   Colorectal cancers, metastatic or non-metastatic: AC, including hereditary forms of AC, carcinoid, sarcoma;
   Pancreatic cancers: AC, including ductal and acinary cancers, papillary, adenosquamous, undifferentiated, tumours of the endocrine pancreas;
   Breast cancers, metastatic or non-metastatic: AC, including invasive ductal, lobular and medullary cancers, tubular, mucinous cancers, Paget-carcinoma, inflammatory carcinoma, ductal and lobular carcinoma in situ;
   Prostate cancers: AC, small cell, SCC;
   Non-small cell lung cancers (NSCLC): SCC, spindle cell carcinoma, AC, bronchioalveolar carcinoma, large cell NSCLC, clear cell NSCLC.

In a preferred embodiment E, with regard to the first, second and third aspect of the invention, compound 1 of formula (I) is selected from the group consisting of
(d) 4-[(3-chloro-4-fluorophenyl)amino]-6-{[4-(N,N-dimethylamino)-1-oxo-2-buten-1-yl]amino}-7-((S)-tetrahydrofuran-3-yloxy)-quinazoline (BIBW2992) or a pharmacologically acceptable salt thereof, preferably the dimaleate salt (d′), and
(k) 4-[(3-chloro-4-fluoro-phenyl)amino]-6-{[4-(homomorpholin-4-yl)-1-oxo-2-buten-1-yl]amino}-7-[(S)-(tetrahydrofuran-3-yl)oxy]-quinazoline,
   the chemotherapeutic agent 2 is selected from the group consisting of docetaxel and paclitaxel,
   and the cancer indication is selected from the group consisting of
   Breast cancers, metastatic or non-metastatic: AC, including invasive ductal, lobular and medullary cancers, tubular, mucinous cancers, Paget-carcinoma, inflammatory carcinoma, ductal and lobular carcinoma in situ;

In a preferred embodiment F, with regard to the first, second and third aspect of the invention, compound 1 of formula (I) is selected from the group consisting of
(d) 4-[(3-chloro-4-fluorophenyl)amino]-6-{[4-(N,N-dimethylamino)-1-oxo-2-buten-1-yl]amino}-7-((S)-tetrahydrofuran-3-yloxy)-quinazoline (BIBW2992) or a pharmacologically acceptable salt thereof, preferably the dimaleate salt (d′),
   the chemotherapeutic agent 2 is selected from the group consisting of irinotecan and oxaliplatin,
   or the chemotherapeutic agent 2 is 5-FU, optionally combined with leucovorin,
   and the cancer indication is selected from the group consisting of
   Colorectal cancers, metastatic or non-metastatic: AC, including hereditary forms of AC, carcinoid, sarcoma.

In a preferred embodiment G, with regard to the first, second and third aspect of the invention, compound 1 of formula (I) is selected from the group consisting of
(d) 4-[(3-chloro-4-fluorophenyl)amino]-6-{[4-(N,N-dimethylamino)-1-oxo-2-buten-1-yl]amino}-7-((S)-tetrahydrofuran-3-yloxy)-quinazoline (BIBW2992) or a pharmacologically acceptable salt thereof, preferably the dimaleate salt (d′), and
(k) 4-[(3-chloro-4-fluoro-phenyl)amino]-6-{[4-(homomorpholin-4-yl)-1-oxo-2-buten-1-yl]amino}-7-[(S)-(tetrahydrofuran-3-yl)oxy]-quinazoline,
   the chemotherapeutic agent 2 is docetaxel, optionally combined with estramustine,
   and the cancer indication is selected from the group consisting of
   Prostate cancers: AC, small cell, SCC, hormone sensitive or hormone refractory prostate cancer.

In a preferred embodiment H, with regard to the first, second and third aspect of the invention, compound 1 of formula (I) is selected from the group consisting of
(d) 4-[(3-chloro-4-fluorophenyl)amino]-6-{[4-(N,N-dimethylamino)-1-oxo-2-buten-1-yl]amino}-7-((S)-tetrahydrofuran-3-yloxy)-quinazoline (BIBW2992),
(k) 4-[(3-chloro-4-fluoro-phenyl)amino]-6-{[4-(homomorpholin-4-yl)-1-oxo-2-buten-1-yl]amino}-7-[(S)-(tetrahydrofuran-3-yl)oxy]-quinazoline,
   the chemotherapeutic agent 2 is selected from the group consisting of
4-[[(7R)-8-(cyclopentyl)-7-ethyl-5,6,7,8-tetrahydro-5-methyl-6-oxo-2-pteridinyl]-amino]-N-3-methoxy-N—(N-methyl-4-piperidinyl)-benzamide (described in WO 2004/076454), and
N-[trans-4-[4-(cyclopropylmethyl)-1-piperazinyl]cyclohexyl]-4-[[(7R)-7-ethyl-5,6,7,8-tetrahydro-5-methyl-8-(1-methylethyl)-6-oxo-2-pteridinyl]amino]-3-methoxy-benzamide (described in WO 2004/076454),
   and the cancer indication is selected from the group consisting of
   Head and neck tumours: SCC, AC, transitional cell cancers, mucoepidermoid cancers, undifferentiated carcinomas;
   Colorectal cancers, metastatic or non-metastatic: AC, including hereditary forms of AC, carcinoid, sarcoma;
   Pancreatic cancers: AC, including ductal and acinary cancers, papillary, adenosquamous, undifferentiated, tumours of the endocrine pancreas;
   Breast cancers, metastatic or non-metastatic: AC, including invasive ductal, lobular and medullary cancers, tubular, mucinous cancers, Paget-carcinoma, inflammatory carcinoma, ductal and lobular carcinoma in situ;
   Prostate cancers: AC, small cell, SCC;
   Gastric cancers: AC, adenosquamous, anaplastic;
   Ovarian cancer;
   Non-small cell lung cancers (NSCLC): SCC, spindle cell carcinoma, AC, bronchioalveolar carcinoma, large cell NSCLC, clear cell NSCLC.

Within any of the embodiments of the invention directed to a method of treatment, is specifically within embodiments A to H mentioned hereinbefore, radiotherapy or radio-immunotherapy can optionally be added as a co-therapy.

It is known that cancer patients carrying activating EGFR mutations in their tumors, i. e. within the tyrosine kinase domain of the EGF receptor, may show increased sensitivity to treatment with EGFR inhibitors. Analogously, cancer patients carrying activating HER2 mutations, e.g. M774_A775insAYVM, in their tumors may show increased sensitivity to treatment with HER2 inhibitors. Both groups of patients as well as a subgroup carrying both activating EGFR and HER2 mutations may show increased sensitivity to treatment with dual inhibitors of erbb1 receptor (EGFR) and erbB2 (Her2/neu).

The presence of specific gain-of-function mutations within the tyrosine kinase domain of the EGF receptor in a subgroup of NSCLC patients has been associated with increased sensitivity to treatment with gefitinib and erlotinib (Lynch, New England Journal Medicine 350, 2129 (2004); Paez, Science 304, 1497 (2004); Pao, Proceedings of the National Academy of Science of the United States 101, 13306 (2004)). In particular, the L858R point mutation (exon 21) as well as deletion/insertion mutations in the ELREA sequence (exon 19) account for the majority of gefitinib responders. A secondary point mutation in exon 20, T790M, is associated with acquired resistance to gefitinib or erlotinib. This mutation is analogous to the T315I mutation identified in CML patients who relapse under imatinib treatment (imatinib resistant patients).

Irreversible inhibitors (e.g., HKI-272 or CL 387,785), in contrast to reversible inhibitors (e.g., gefitinib), are able to inhibit proliferation and EGF-induced EGFR phosphorylation in cell lines expressing double mutant EGF receptors (Kwak, Proceedings of the National Academy of Science of the United States 102, 7665 (2005) and Kobayashi, New England Journal Medicine 352, 786 (2005)).

Any aspect of the present invention therefore includes, as a sub-aspect, optional pre-selection of cancer patients for an EGFR mutation in the tyrosine kinase domain of the EGF receptor as well as pre-selection of cancer patients for an HER2 mutation. The EGFR mutations preferably relevant in this context are selected from the group consisting of the L858R and L861 point mutations in the activation loop (exon 21), in-frame deletion/insertion mutations in the ELREA sequence (exon 19), substitutions in G719 situated in the nucleotide binding loop (exon 18), activating mutations in the extracellular domain of the EGF receptor such as EGFR vIII displaying exon 2-7 deletions, the T790M point mutation in exon 20, exon 20 insertions such as D770_N771insNPG, and double mutants such as the combined L858R/T790M mutation and the exon-19-del/T790M. The HER2 mutation preferably relevant in in this context is the M774_A775insAYVM mutation.

Methods for detecting mutations in the tyrosine kinase domain of the EGF receptor are kown in the art, several corresponding diagnostic tools are approved by the FDA and commercially available, e.g. an assay for the detection of epidermal growth factor receptor mutations in patients with non-small cell lung cancer (Genzyme Corp.; see also Journal of Clinical Oncology, 2006 ASCO Annual Meeting Proceedings (Post-Meeting Edition). Vol 24, No 18S (June 20 Supplement), 2006: Abstract 10060).

Any of the embodiments (1) to (32), A, B, C, D, E, F, G and H of the invention mentioned hereinbefore defining compound 1 of formula (I), chemotherapeutic agents 2 and cancer indications applies accordingly to the optional sub-aspect of pre-selection of cancer patients for an activating EGFR mutation in the tyrosine kinase domain of the EGF receptor and/or pre-selection of cancer patients for an activating HER2 mutation. Treatment of EGFR mutant cancer patients with the compounds of formula (I) may allow a responce in cancer patients with acquired or persistent resistance to gefitinib or erlotinib treatment. Treatment of cancer patients carrying an activating HER2 mutant in their tumors with the compounds of formula (I) may allow a responce in cancer patients with acquired or persistent resistance to certain chemotherapeutics such as e. g. lapatinib or herceptin.

Most preferred cancer indications with EGFR or HER2 mutations relevant in connection with the sub-aspect of patient pre-selection for mutations are selected from the group consisting of
Head and neck tumours: SCC, AC, transitional cell cancers, is mucoepidermoid cancers, undifferentiated carcinomas;
Colorectal cancers, metastatic or non-metastatic: AC, including hereditary forms of AC, carcinoid, sarcoma;
Pancreatic cancers: AC, including ductal and acinary cancers, papillary, adenosquamous, undifferentiated, tumours of the endocrine pancreas;
Breast cancers, metastatic or non-metastatic: AC, including invasive ductal, lobular and medullary cancers, tubular, mucinous cancers, Paget-carcinoma, inflammatory carcinoma, ductal and lobular carcinoma in situ;
Prostate cancers: AC, small cell, SCC;
Gastric cancers: AC, adenosquamous, anaplastic;
Ovarian cancer;
Non-small cell lung cancers (NSCLC): SCC, spindle cell carcinoma, AC, bronchioalveolar carcinoma, large cell NSCLC, clear cell NSCLC,
   but especially
Non-small cell lung cancers (NSCLC): SCC, spindle cell carcinoma, AC, bronchioalveolar carcinoma, large cell NSCLC, clear cell NSCLC, especially metastatic, second line patients who have failed at least one prior chemotherapy regimen or 3rd/4th line patients who have received Tarceva or Iressa for at least 12 weeks and then failed,
   preferably to be treated by administration of a compound 1 selected from the group consisting of:
(a) 4-[(3-chloro-4-fluorophenyl)amino]-6-{[4-(N,N-dimethylamino)-1-oxo-2-buten-1-yl]amino}-7-cyclobutyloxy-quinazoline,
(b) 4-[(3-chloro-4-fluorophenyl)amino]-6-{[4-(N,N-dimethylamino)-1-oxo-2-buten-1-yl]amino}-7-cyclopentyloxy-quinazoline,
(c) 4-[(3-chloro-4-fluorophenyl)amino]-6-{[4-(N,N-dimethylamino)-1-oxo-2-buten-1-yl]amino}-7-((R)-tetrahydrofuran-3-yloxy)-quinazoline,
(d) 4-[(3-chloro-4-fluorophenyl)amino]-6-{[4-(N,N-dimethylamino)-1-oxo-2-buten-1-yl]amino}-7-((S)-tetrahydrofuran-3-yloxy)-quinazoline (BIBW2992),
(e) 4-[(3-chloro-4-fluorophenyl)amino]-6-{[4-(N,N-dimethylamino)-1-oxo-2-buten-1-yl]amino}-7-(tetrahydropyran-4-yloxy)-quinazoline,
(f) 4-[(3-chloro-4-fluorophenyl)amino]-6-{[4-(N,N-dimethylamino)-1-oxo-2-buten-1-yl]amino}-7-[(tetrahydrofuran-2-yl)methoxy]-quinazoline,
(g) 4-[(3-chloro-4-fluorophenyl)amino]-6-{[4-(N,N-dimethylamino)-1-oxo-2-buten-1-yl]amino}-7-[(tetrahydrofuran-3-yl)methoxy]-quinazoline,
(h) 4-[(R)-(1-phenyl-ethyl)amino]-6-{[4-(N,N-dimethylamino)-1-oxo-2-buten-1-yl]amino}-7-cyclopropylmethoxy-quinazoline,
(i) 4-[(3-chloro-4-fluorophenyl)amino]-6-{[4-(morpholin-4-yl)-1-oxo-2-buten-1-yl]amino}-7-[(tetrahydrofuran-2-yl)methoxy]-quinazoline,
(j) 4-[(3-chloro-4-fluorophenyl)amino]-6-{[4-(N,N-dimethylamino)-1-oxo-2-buten-1-yl]amino}-7-[(S)-(tetrahydrofuran-2-yl)methoxy]-quinazoline,
(k) 4-[(3-chloro-4-fluoro-phenyl)amino]-6-{[4-(homomorpholin-4-yl)-1-oxo-2-buten-1-yl]amino}-7-[(S)-(tetrahydrofuran-3-yl)oxy]-quinazoline, and
(r) 4-[(3-chloro-4-fluoro-phenyl)amino]-6-{[4-(dimethylamino)-1-oxo-2-buten-1-yl]amino}-7-cyclopropylmethoxy-quinazoline,
   or a pharmaceutically acceptable salt thereof,
   and co-aministration of a chemotherapeutic agent 2 selected from the group consisting of
   gefitinib, erlotinib, HKI-272, lapatinib, carboplatin, cisplatin, gemcitabine, docetaxel, paclitaxel and pemetrexed.

### Method of Treatment:

The method of treatment according to the invention comprises administration of therapeutically effective amounts of:
(1) a compound 1 of formula (I); and
(2) at least a further chemotherapeutic agent 2;
   to a patient in need thereof, wherein the active ingredients are administered orally, enterically, transdermally, intravenously, peritoneally or by injection, preferably orally, optionally in combination with radiotherapy, radio-immunotherapy and/or tumour resection by surgery.

In a further embodiment the invention relates to a method for the treatment of cancer, which method comprises simultaneous, separate or sequential co-administration of effective amounts of:
(1) a compound 1 of formula (I); and
(2) at least a further chemotherapeutic or naturally occurring, semi-synthetic or synthetic therapeutic agent 2;
   in the form of a combined preparation optionally adapted for a co-treatment with radiotherapy or radio-immunotherapy, to a person in need of such treatment.

The term “therapeutically effective amount” shall mean that amount of a drug or pharmaceutical agent that will elicit the biological or medical response of a tissue, system, animal or human that is being sought by a researcher or clinician.

In accordance with the present invention, the elements of the combination of 1 and 2 may be administered by oral (including buccal or sublingual), enterical, parenteral (e.g., intramuscular, intraperitoneal, intravenous, transdermal or subcutaneous injection, or implant), nasal, vaginal, rectal, or topical (e.g. inhalative) routes of administration and may be formulated, alone or together, in suitable dosage unit formulations containing conventional non-toxic pharmaceutically acceptable carriers, adjuvants and vehicles appropriate for each route of administration.

In a preferred embodiment the element 1 of the combination in accordance with the invention is administered orally, enterically, transdermally, intravenously, peritoneally or by injection, preferably orally.

### Dosages/Compound 1:

In one embodiment the invention relates to the method of treatment described above, characterised in that a compound 1 of formula (I), or its polymorph, metabolite, hydrate, solvate, an individual optical isomer, mixtures of the individual enantiomers or racemates thereof, or a pharmaceutically acceptable salt thereof, is administered intermittent or in a daily dosage such that the plasma level of the active substance preferably lies between 10 and 5000 nM for at least 12 hours of the dosing interval.

The compounds of formula (I) may be administered to the human patient in a daily dose of 0.01-4 mg/kg of body weight (bw), preferably 0.1-2 mg/kg, particularly preferred in a dose of 0.2-1.3 mg/kg bw. For oral treatment the compounds of formula (I) may be administered daily in a total dose of 10, 20, 30, 40, 50, 60, 70, 100, 200, or 300 mg, optionally divided into multiple doses, e.g. 1 to 3 doses to be administered through the day. Preferably the oral daily dose is administered only once a time. These doses can be applied with any of the compounds of formula (I), e.g. with BIBW2992 or an equivalent dose of BIBW2992MA₂ containing respective amounts of the active base component. Especially for higher doses periods of treatment should alternate with periods of recovery, without administering the active of formula (I). For instance, treatment could follow a “7 day on-7 day off”, a “14 day on-14 day off”, a “21 day on 7 day off” or a continuous dosing schedule. “On-off” time periods can be chosen shorter, especially if higher doses are administered, or individually adapted to the needs of the patient. The dosage for intravenous use of a compound of formula (I), e.g. of BIBW2992MA₂ may be 1-1000 mg, preferably 5-300 mg, particularly preferred 10-100 mg (dosages refer to the base form BIBW2992), either given as a bolus or, especially if higher doses are applied, as a slow intravenous infusion over several hours, e.g. over about 1, 2, 4, 6, 10, 12 or 24 hours.

However, it may optionally be necessary to deviate from the amounts specified, depending on the body weight or method of administration, the individual response to the medication, the nature of the formulation used and the time or interval over which it is administered. Thus, in some cases, it may be sufficient to use less than the minimum quantity specified above, while in other cases the upper limit specified will have to be exceeded. When large amounts are administered it may be advisable to spread them over the day in a number of single doses.

### Dosages/Chemotherapeutic Agents 2:

Dosages and treatment schedules for the individual chemotherapeutic agents 2 are known in the art and may be applied analogously within the invention. Depending on the individual activity of the specific combination dosage of the chemotherapeutic agents 2 may be reduced, e.g. may vary in the range of 1/1 to 1/20 of the dosages described in the prior art.

For patients with metastatic breast cancer the combination with docetaxel may be given at a dose between 55 mg/m² and 100 mg/m² and most specifically at a dose of 60 to 75 mg/m² in administration schedule of once every 21 days. In a weekly administration schedule the dose of docetaxel may be lowered.

A similar dose range of docetaxel will be used in the treatment of hormone-refractory prostate cancer. In this case docetaxel is administered together with daily prednisone and/or with the administration of estramustine. The dose of estramustine is 14 mg per kg of body weight given in 3 or 4 divided doses daily. Most patients are treated at a dose range between 10 and 16 mg/kg body weight.

Docetaxel is also used in the treatment of non-small cell lung cancer at similar doses and schedules.

In patients with metastatic breast cancer, the administration of paclitaxel is at a dose of up to 175 mg/m² over 3 hours every 3 weeks. In a weekly administration schedule paclitaxel dose may be lower. In an adjuvant setting, paclitaxel will be administered at doses up to 175 mg/m² over 3 hours every 3 weeks sequentially to a combination with a doxorubicin-containing chemotherapy (four courses of doxorubicin and cyclophosphamide were used).

For patients with non-small cell lung cancer the recommended dose of paclitaxel is 135 mg/m² IV over 24 hours every 3 weeks. The administration of paclitaxel is followed by cisplatin at 75 mg/m². Another option is the combination of paclitaxel with carboplatin.

In patients with ovarian carcinoma, paclitaxel is used at a dose of 175 mg/m² IV over 3 hours followed by cisplatin at 75 mg/m² or at a dose of 135 mg/m² over 24 hours followed by cisplatin at a dose of 75 mg/m². Paclitaxel can also be combined with carboplatin. This cycle will be repeated every 3 weeks. Another treatment schedule in the more advanced disease setting is the administration of paclitaxel at either 135 or 175 mg/m² IV over 3 hours every 3 weeks.

Carboplatin is administered as a single agent in recurrent ovarian carcinoma at a dose of 360 mg/m² IV on day 1 every 4 weeks. In advanced ovarian carcinoma it is used at a dose of 300 mg/m² on day 1 every 4 weeks for six cycles together with cyclophosphomide 600 mg/m² on day 1 every four weeks for 6 cycles. Carboplatin is also used in combination with paclitaxel for the treatment of advanced ovarian cancer and advanced non-small cell lung cancer.

In patients with breast cancer and colorectal cancer, the administration of capecitabine is used at a dose of up to 1250 mg/m² twice daily for 2 weeks followed by a 1-week rest before repating this 3-week regimen. Such a dose will also be used in the adjuvant treatment of colorectal cancer for a total of eight 3-week cycles. When combining with drugs like docetaxel dose reductions according to actually experienced side effects may become necessary.

In patients with metastatic breast cancer, gemcitabine at a dose of 1250 mg/m² over 30 minutes on days 1 and 8 of each 21-day treatment cycle will be used in combination with paclitaxel. Paclitaxel should be administered at 175 mg/m² as a 3-hour infusion before the administration of gemcitabine on day 1.

Gemcitabine is also used for the treatment of pancreatic cancer at a dose of up to 1000 mg/m² over 30 minutes once weekly for up to 7 weeks (or until toxicity necessitates reducing or holding the dose) followed by a week of rest. Subsequent cycles will be administration for 3 consecutive weeks every 4 weeks.

In non-small cell lung cancer, gemcitabine is used in two schedules. In the first schedule, gemcitabine is administered at 1000 mg/m² over 30 minutes on days 1, 8, and 15 every 4 weeks. Cisplatin is administered at 100 mg/m² IV on day 1 after the infusion of gemcitabine. In another schedule gemcitabine is administered at 1250 mg/m² IV over 30 minutes on days 1 and 8 every 3 weeks. Cisplatin should be administered at 100 mg/m² IV on day 1.

Trastuzumab is used either single agent or in combination with paclitaxel for the treatment of HER2-positive breast cancer. Trastuzumab is recommended at an initial loading dose of 4 mg/kg as a 90-minute infusion. The weekly recommended maintenance dose is 2 mg/kg as a 30 minute infusion. Additional dose schedules are under consideration.

In combination with a dosing schedule (FOLFOX4) for the treatment of colorectal cancer, oxaliplatin may be administered on day 1 in a dose of up to 85 mg/m² (in infusions of up to 2 hours or more). Leucovorin in this schedule may be up to 200 mg/m² (in infusions of up to 2 hours or more) while fluorouracil may used in doses up to 400 mg/m² (bolus) followed by infusion of 600 mg/m² over 22 hours. On day 2, the administration will be leucovorin may be up to 200 mg/m² (in infusions of up to 2 hours or more) while fluorouracil may used in doses up to 400 mg/m² (bolus) followed by infusion of 600 mg/m² over 22 hours. Such an regimen may be repeated every 2 weeks. Other treatment schedules based on variations of administration lengths of oxaliplatin, leucovorin and fluorouracil may also apply.

Also in the treatment of colorectal cancer other schedules may be used. These include irinotecan 125 mg/m² as a 90 minute infusion, leucovorin as a 20 mg/m² (15 minute bolus or IV push) followed by fluorouracil 500 mg/m² (bolus every week×4). This schedule will be repeated every 6 weeks. Another treatment schedule is the administration of irinotecan 180 mg/m² as a 90 minute infusion (day 1, 15, 29), leucovorin at 200 mg/m² over 2 hours (days 1, 2, 15, 16, 29, 30), and fluorouracil as 400 mg/m² bolus followed by an infusion of 600 mg/m² over 22 hours (both on days 1, 2, 15, 16, 29, 30). This schedule will be repeated on day 43. Other treatment schedules based on variations of administration lengths of irinotecan, leucovorin and fluorouracil may also apply.

Irinotecan may also applied for colorectal cancer in a dosing schedule of 125 mg/m² over 90 minutes on days 1, 8, 15, 22 followed by 2 week rest before repeating the schedule. Another option would be dosing of irinotecan at 350 mg/m² over 90 minutes every 3 weeks.

Another treatment schedule for colorectal cancer may be administered by combination with leucovorin at 200 mg/m² (2-hour infusion) followed by fluorouracil 400 mg/m² (bolus) and 600 mg/m² (22 hour infusion) at day 1. On day 2 this schedule is repeated. Such a schedule is repeated every 2 weeks. Other treatment schedules based on variations of administration lengths of leucovorin and fluorouracil may also apply.

However, it may optionally be necessary to deviate from the amounts specified, depending on the body weight or method of administration, the individual response to the medication, the nature of the formulation used and the time or interval over which it is administered. Thus, in some cases, it may be sufficient to use less than the minimum quantity specified above, while in other cases the upper limit specified will have to be exceeded. When large amounts are administered it may be advisable to spread them over the day in a number of single doses.

### Dosages/Radiotherapy or Radio-Immunotherapy:

Dosages and treatment schedules for radiotherapy and radio-immunotherapy are known in the art and may be applied analogously within the invention. Depending on the individual activity of the specific combination with compound 1 and, optionally, chemotherapeutic agent 2, dosage of the radiotherapy and radio-immunotherapy component may be reduced, e.g. may vary in the range of 1/1 to 1/20 of the dosages described in the prior art.

### Pharmaceutical Compositions:

As used herein, the term “composition” is intended to encompass a product comprising the specified ingredients in the specified amounts, as well as any product which results, directly or indirectly, from a combination of the specified ingredients in the specified amounts. The amount of pharmaceutically active compound in each case should be in the range from 0.1-90 wt. %, preferably 0.5-50 wt. % of the total composition, i.e. in amounts which are sufficient to achieve the dosage ranges given hereinbefore. The doses specified may, if necessary, be given several times a day.

As already mentioned before, within the meaning of the present invention, the components 1 and 2 of the composition for a combination therapy may be administered separately (which implies that they are formulated separately) or together (which implies that they are formulated together). Hence, the administration of one element of the combination of the present invention may be prior to, concurrent to, or subsequent to the administration of the other element of the combination.

One embodiment of the invention relates to a pharmaceutical combination preparation kit for the treatment of cancer diseases, comprising
(i) a first compartment containing a pharmaceutical composition comprising a therapeutically effective amount of a compound 1 of formula (I), and
(ii) a second containment containing a pharmaceutical composition comprising at least a further chemotherapeutic agent 2 in a therapeutically effective amount,
said kit being optionally adapted for a co-treatment with radiotherapy or radio-immunotherapy.

In a preferred embodiment the invention relates to a pharmaceutical combination preparation kit, wherein the formulation of the compound 1 of formula (I) in accordance with the present invention is for oral administration.

The pharmaceutical compositions for the administration of the components 1 and 2 of this invention may conveniently be presented in dosage unit form and may be prepared by any of the methods well known in the art of pharmacy. All methods include the step of bringing the active ingredient into association with the carrier which is constituted of one or more accessory ingredients. In general, the pharmaceutical compositions are prepared by uniformly and intimately bringing the active ingredients into association with a liquid carrier or a finely divided solid carrier or both, and then, if necessary, shaping the product into the desired dosage form. In the pharmaceutical compositions the active compounds are included in an amount sufficient to produce the desired pharmacologic effect.

Suitable excipients may be, for example, water, pharmaceutically acceptable organic solvents, such as paraffins (e.g. petroleum fractions), oils of vegetable origin (e.g. groundnut or sesame oil), mono- or polyfunctional alcohols (e.g. ethanol or glycerol), carriers such as e.g. natural mineral powders (e.g. kaolin, clays, talc, chalk), synthetic mineral powders (e.g. highly dispersed silica and silicates), sugar (e.g. glucose, lactose and dextrose), emulsifiers (e.g. lignin, spent sulphite liquors, methylcellulose, starch and polyvinylpyrrolidone) and lubricants (e.g. magnesium stearate, talc, stearic acid and sodium lauryl sulphate).

The preparations are administered in the usual way, preferably by oral or transdermal route, particularly preferably by oral route. When administered orally the tablets may, of course, contain additives, such as e.g. sodium citrate, calcium carbonate and dicalcium phosphate together with various additives, such as starch, preferably potato starch, gelatine and the like, in addition to the abovementioned carriers. Lubricants such as magnesium stearate, sodium laurylsulphate and talc may also be used to form tablets. In the case of aqueous suspensions the active substances may be combined with various flavour enhancers or colourings in addition to the abovementioned excipients. For parenteral use, solutions of the active substances may be prepared using suitable liquid carrier materials.

The pharmaceutical compositions containing the active ingredients 1 and 2, separately or together, that are suitable for oral administration may be in the form of discrete units such as hard or soft capsules, tablets, troches or lozenges, each containing a predetermined amount of the active ingredients, or in the form of a dispersible powder or granules, or in the form of a solution or a suspension in an aqueous liquid or non-aqueous liquid, or in the form of syrups or elixirs, or in the form of an oil-in-water emulsion or a water-in-oil emulsion.

Dosage forms intended for oral use may be prepared according to any method known to the art for the manufacture of pharmaceutical formulations and such compositions. The excipients used may be, for example: (a) inert diluents such as mannitol, sorbitol, calcium carbonate, pregelatinized starch, lactose, calcium phosphate or sodium phosphate; (b) granulating and disintegrating agents, such as povidone, copovidone, hydroxypropylmethylcellulose, corn starch, alginic acid, crospovidone, sodiumstarchglycolate, croscarmellose, or polacrilin potassium; (c) binding agents such as microcrystalline cellulose or acacia; and (d) lubricating agents such as magnesium stearate, stearic acid, fumaric acid or talc.

Coated tablets may be prepared accordingly by coating cores produced analogously to the tablets with substances normally used for tablet coatings, for example collidone or shellac, gum arabic, talc, titanium dioxide or sugar. To achieve delayed release or prevent incompatibilities the core may also consist of a number of layers. Similarly the tablet coating may consist of a number of layers to achieve delayed release, possibly using the excipients mentioned above for the tablets.

Capsules containing one or more active substances or combinations of active substances may for example be prepared by mixing the active substances with inert carriers such as lactose or sorbitol and packing them into gelatine capsules. In some cases, formulations for oral use may be in the form of hard gelatin or HPMC (hydroxypropylmethylcellulose) capsules wherein the active ingredients 1 or 2, separately or together, is mixed with an inert solid diluent, for example pregelatinized starch, calcium carbonate, calcium phosphate or kaolin, or dispensed via a pellet formulation. They may also be in the form of soft gelatin capsules wherein the active ingredient is mixed with water or an oil medium, for example peanut oil, liquid paraffin, medium chain triglycerides or olive oil.

The tablets, capsules or pellets may be uncoated or they may be coated by known techniques to delay disintegration and absorption in the gastrointestinal tract and thereby provide a delayed action or sustained action over a longer period. For example, a time delay material such as celluloseacetate phtalate or hydroxypropylcellulose acetate succinate or sustained release material such as ethylcellulose or ammoniomethacrylate copolymer (type B) may be employed.

Liquid dosage forms for oral administration in accordance with the present invention include pharmaceutically acceptable emulsions, solutions, suspensions, syrups, and elixirs containing inert diluents commonly used in the art, such as water. Besides such inert diluents, compositions can also include adjuvants, such as wetting agents, emulsifying and suspending agents, and sweetening, flavoring, perfuming and preserving agents.

Syrups or elixirs containing the active substances or combinations thereof according to the invention may additionally contain a sweetener such as saccharin, cyclamate, glycerol or sugar and a flavour enhancer, e.g. a flavouring such as vanillin or orange extract. They may also contain suspension adjuvants or thickeners such as sodium carboxymethyl cellulose, wetting agents such as, for example, condensation products of fatty alcohols with ethylene oxide, or preservatives such as p-hydroxybenzoates.

Aqueous suspensions in accordance with the present invention normally contain the active materials 1 and 2, separately or together, in admixture with excipients suitable for the manufacture of aqueous suspensions. Such excipients may be (a) suspending agents such as hydroxy ethylcellulose, sodium carboxymethylcellulose, methylcellulose, hydroxypropylmethylcellulose, sodium alginate, polyvinylpyrrolidone, gum tragacanth and gum acacia; (b) dispersing or wetting agents which may be (b.1) a naturally-occurring phosphatide such as lecithin, (b.2) a condensation product of an alkylene oxide with a fatty acid, for example, polyoxyethylene stearate, (b.3) a condensation product of ethylene oxide with a long chain aliphatic alcohol, for example heptadecaethyleneoxycetanol, (b.4) a condensation product of ethylene oxide with a partial ester derived from a fatty acid and a hexitol such as polyoxyethylene sorbitol monooleate, or (b.5) a condensation product of ethylene oxide with a partial ester derived from a fatty acid and a hexitol anhydride, for example polyoxyethylene sorbitan monooleate. The aqueous suspensions may also contain: one or more preservatives, for example, ethyl or n-propyl p-hydroxybenzoate; one or more coloring agents; one or more flavoring agents; and one or more sweetening agents, such as sucrose or saccharin.

Oily suspensions in accordance with the present invention may be formulated by suspending the active ingredients 1 and 2, separately or together, in a vegetable oil, for example arachis (peanut) oil, olive oil, sesame oil or coconut oil, or in a mineral oil such as liquid paraffin. The oily suspensions may contain a thickening agent, for example beeswax, hard paraffin or cetyl alcohol. Sweetening agents and flavoring agents may be added to provide a palatable oral preparation. These compositions may be prepared by the addition of an antioxidant such as ascorbic acid.

Dispersible powders and granules are suitable formulations for the preparation of an aqueous suspension in accordance with the present invention. In these formulations the active ingredients 1 and 2 are present, separately or together, in admixture with a dispersing or wetting agent, a suspending agent and one or more preservatives. Suitable examples of dispersing or wetting agents, suspending agents and preservatives are those already mentioned hereinbefore. Additional excipients such as, for example, sweetening, flavouring and colouring agents may also be present. Suitable examples of excipients are those already mentioned hereinbefore.

The pharmaceutical compositions of the invention may also be in the form of oil-in-water emulsions. The oily phase may be a vegetable oil such as olive oil or arachis (peanut) oil, or a mineral oil such as liquid paraffin or a mixture thereof. Suitable emulsifying agents may be (a) naturally-occurring gums such as gum acacia and gum tragacanth, (b) naturally-occurring phosphatides such as soybean and lecithin, (c) esters or partial esters derived from fatty acids and hexitol anhydrides, for example sorbitan monooleate, (d) condensation products of said partial esters with ethylene oxide, for example polyoxyethylene sorbitan monooleate. The emulsions may also contain sweetening and flavouring agents.

Syrups and elixirs in accordance with the present invention may be formulated with sweetening agents, for example glycerol, propylene glycol, sorbitol or sucrose. Such formulations may also contain a preservative and flavoring and coloring agents.

The pharmaceutical compositions containing 1 and 2, separately or together, may be in the form of a sterile injectable aqueous or oleagenous suspension or solution. The suspension may be formulated according to known methods using those suitable dispersing or wetting agents and suspending agents which have been mentioned hereinbefore. A suitable sterile injectable preparation may also be a sterile injectable solution or suspension in a non toxic parenterally-acceptable diluent or solvent, for example a solution in 1,3-butane-diol. Examples of suitable acceptable vehicles and solvents that may be employed are water, Ringer's solution and an isotonic sodium chloride solution. In addition, sterile, fixed oils may conventionally be employed as a solvent or suspending medium. For this purpose any bland fixed oil may be employed, including synthetic mono- or diglycerides. In addition, fatty acids such as oleic acid find use in the preparation of injectables in accordance with the present invention. Preparations for parenteral administration according to the present invention containing 1 and 2, separately or together, include sterile aqueous or non-aqueous solutions, suspension, or emulsions.

Solutions for injection and infusion are prepared in the usual way, e.g. with the addition of preservatives such as p-hydroxybenzoates, or stabilisers such as alkali metal salts of ethylenediamine tetraacetic acid, optionally using emulsifiers and/or dispersants, while if water is used as the diluent organic solvents may optionally be used as solubilisers or auxiliary solvents, and transferred into injection vials or ampoules or infusion bottles.

Examples of suitables non-aqueous solvents or vehicles for the preparations in accordance with the present invention are propylene glycol, polyethylene glycol, vegetable oils, such as olive oil and corn oil, gelatin, and injectable organic esters such as ethyl oleate. Such dosage forms may also contain adjuvants such as preserving, wetting, emulsifying, and dispersing agents. They may be sterilized by, for example, by filtration through a bacteria-retaining filter, by incorporating sterilizing agents into the compositions, by irradiating the compositions, or by heating the compositions. They may also be manufactured in the form of sterile solid compositions which can be reconstituted in sterile water, or some other sterile injectable medium immediately before use.

The elements 1 and 2 of the combination of this invention may also be administered in the form of suppositories for rectal administration. Such compositions can be prepared by mixing the active ingredient with a suitable non-irritating excipient which is solid at ordinary temperatures but liquid at the rectal temperature and will therefore melt in the rectum to release the active ingredient. Such materials are cocoa butter, hard fat, and polyethylene glycols.

Compositions for buccal, nasal or sublingual administration in accordance with the present invention may be prepared with standard excipients well known in the art.

For topical administration, the elements 1 and 2 of the combination of this invention may be formulated, separately or together, in liquid or semi-liquid preparations. Examples of suitable preparations are: liniments, lotions, applications; oil-in-water or water-in-oil emulsions such as creams, ointments, jellies or pastes, including tooth-pastes; solutions or suspensions such as drops.

Suitable suppositories may be made for example by mixing with carriers provided for this purpose, such as neutral fats or polyethyleneglycol or the derivatives thereof.

The dosage of the active ingredients in the compositions in accordance with the present invention may be varied, although the amount of the active ingredients 1 and 2 shall be such that a suitable dosage form is obtained. Hence, the selected dosage and the selected dosage form shall depend on the desired therapeutic effect, the route of administration and the duration of the treatment. Suitable dosage ranges for the combination are from the maximal tolerated dose for the single agent to lower doses, e.g. to one tenth of the maximal tolerated dose.

The following Examples serve to illustrate the invention without restricting it:

### Example 1

### Gastric Cancer N87 Xenografts in Mice

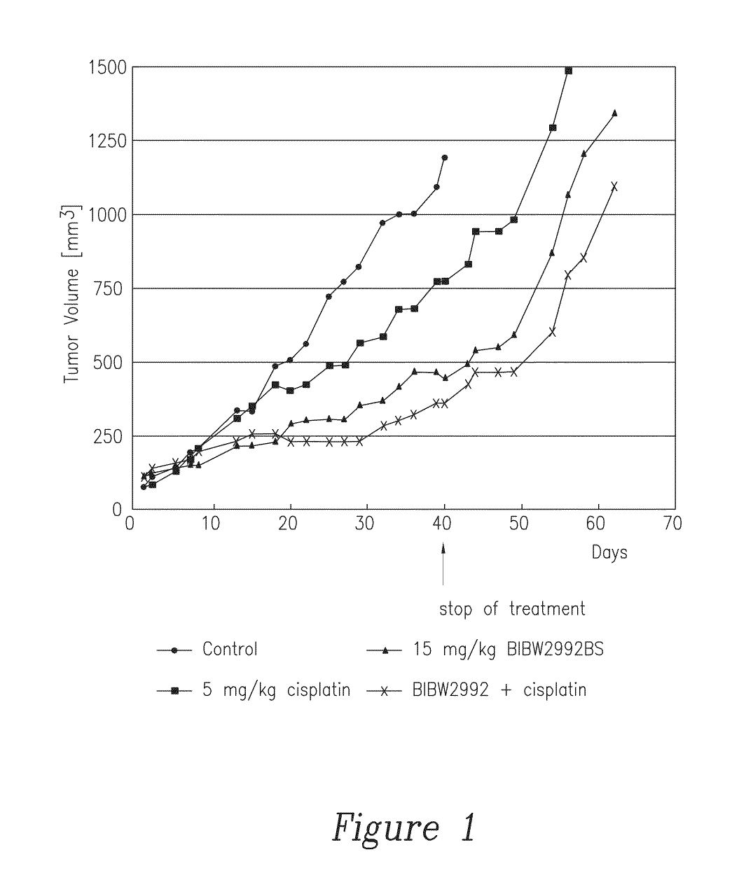

Results of an experiment comparing daily treatment of gastric cancer N87 xenografts in mice with BIBW2992 alone (15 mg/kg), once weekly cis-platin alone (5 mg/kg) and the combination of BIBW2992/cis-platin (15 mg/kg/(5 mg/kg) are shown in FIG. 1 (Appendix). Tretment was stopped on day 40 when the controls were euthanized. While no advantage compared to BIBW 2992 single treatment was achieved the data shows that no antagonistic activity was observed with this combination.

### Example 2

### Ovarian Carcinoma SKOV-3 Xenografts in Mice

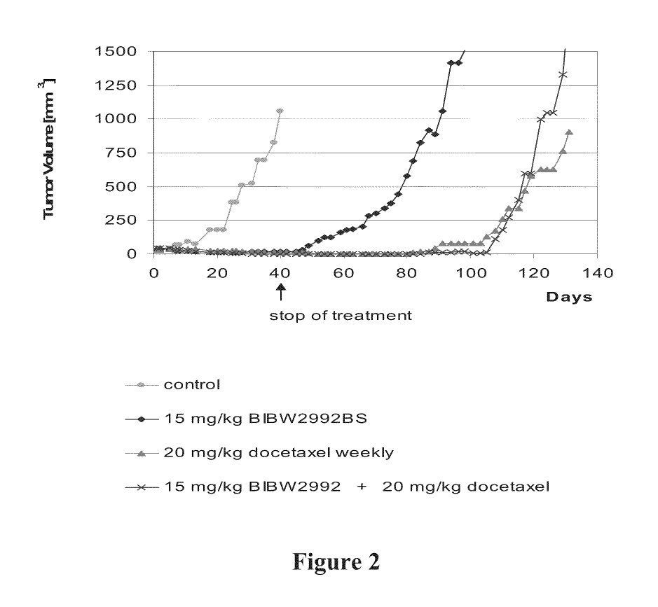

Results of an experiment comparing treatment of ovarian cancer SKOV-3 xenografts in mice with daily administration of BIBW2992 alone (15 mg/kg), once weekly docetaxel alone (20 mg/kg) and the combination of BIBW2992/docetaxel (15 mg/kg/(20 mg/kg) are shown in FIG. 2 (Appendix). Treatment was stopped on day 40, when the controls were euthanized. While no advantage compared to docetaxel single treatment was achieved the data shows that no antagonistic activity was observed with this combination.

### Example 3

### Ovarian Carcinoma SKOV-3 Xenografts in Mice

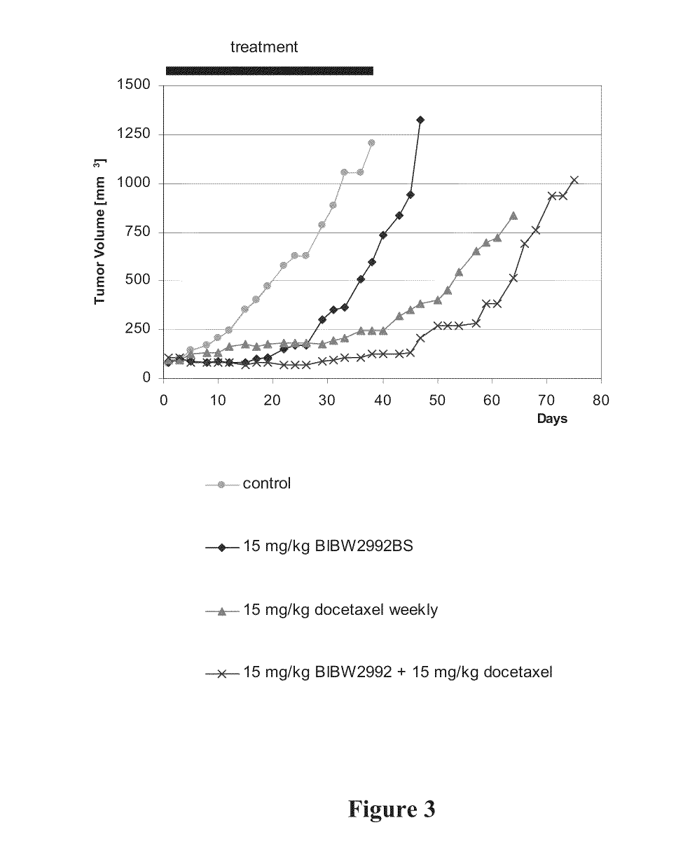

Results of an experiment comparing daily treatment of ovarian cancer SKOV-3 xenografts in mice with BIBW2992 alone (15 mg/kg), docetaxel alone (15 mg/kg) once weekly and the combination of BIBW2992/docetaxel (15 mg/kg/(15 mg/kg) are shown in FIG. 3 (Appendix). Treatment was stopped on day 42. While only a slight advantage compared to docetaxel single treatment was achieved the data shows that no antagonistic activity was observed with this combination.

### Example 4

### Ovarian Cancer SKOV-3 Xenografts in Mice

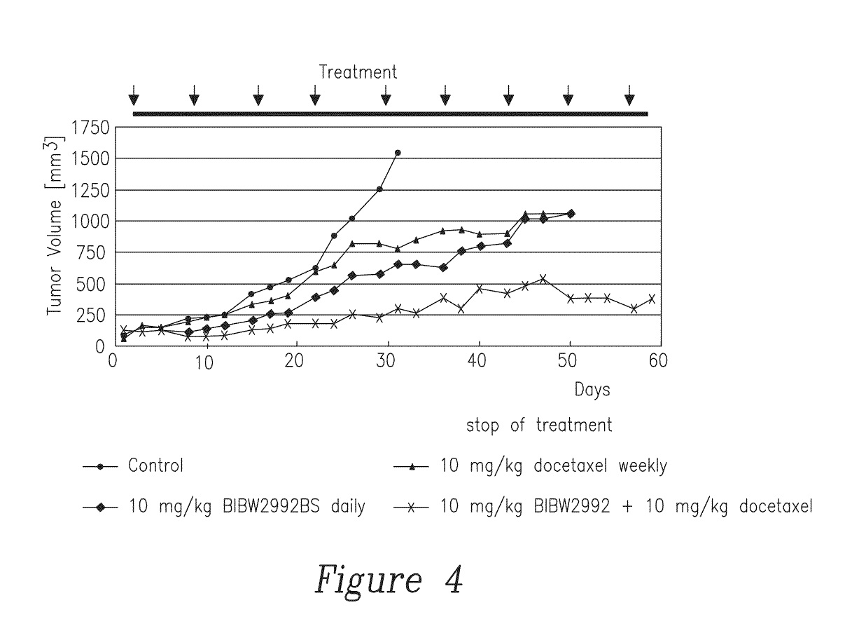

Results of an experiment comparing daily treatment of ovarian cancer SKOV-3 xenografts in mice with BIBW2992 alone (10 mg/kg), once weekly treatment with docetaxel alone (10 mg/kg) and the combination of BIBW2992/docetaxel (10 mg/kg/(10 mg/kg) are shown in FIG. 4 (Appendix). Animals were treted until the end of the experiment. Combination treatment significantly delayed tumor growth compared to single agent treatment. Therefore a clear advantage of combination treatment could be shown compared to single agent treatment with BIBW 2992 or docetaxel.

### Example 5

### Ovarian Carcinoma SKOV-3 Xenografts in Mice

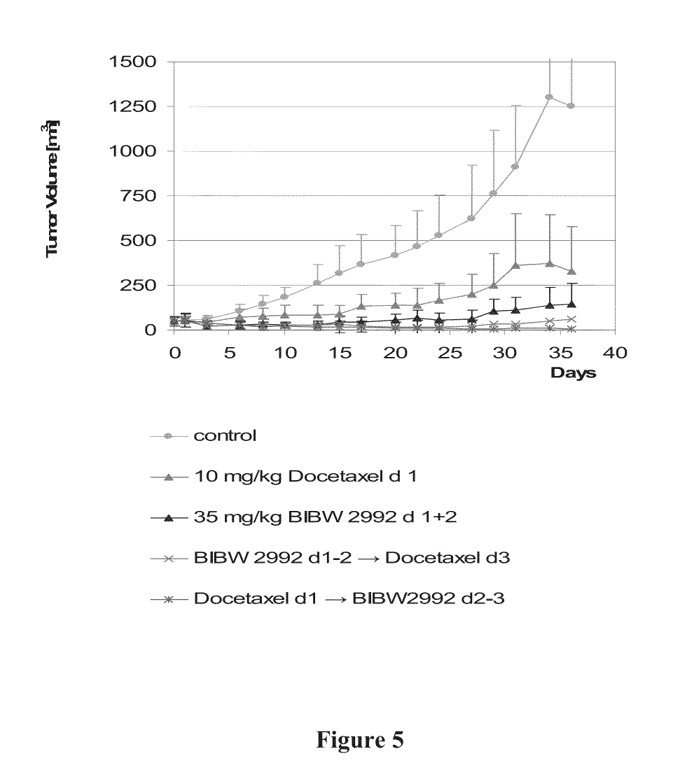

Results of an experiment comparing treatment of ovarian cancer SKOV-3 xenografts in mice with BIBW2992 alone (35 mg/kg), administered twice weekly on two consecutive days, docetaxel alone (10 mg/kg), given once weekly and the pulsatile combination of BIBW2992 and docetaxel are shown in FIG. 5 (Appendix). For the combinations BIBW 2992 was administered for two consecutive days (day 1-2) followed by a single admiinistration of docetaxel on day 3 or docetaxel was given on day 1 followed by BIBW 2992 on two consecutive days (day 2-3). Treatment cycles were repeated weekly throughout the experiment. This study clearly shows that the combination treatments with BIBW 2992 and docetaxel resulted in better anti-tumor effects than either drug alone. Furthermore, the data demonstrate that docetaxel administration followed by BIBW 2992 on two consecutive days results in better and persistent anti-tumor activity than the inverse schedule.

### Example 6

### MDA-453 Breast Xenografts in Mice

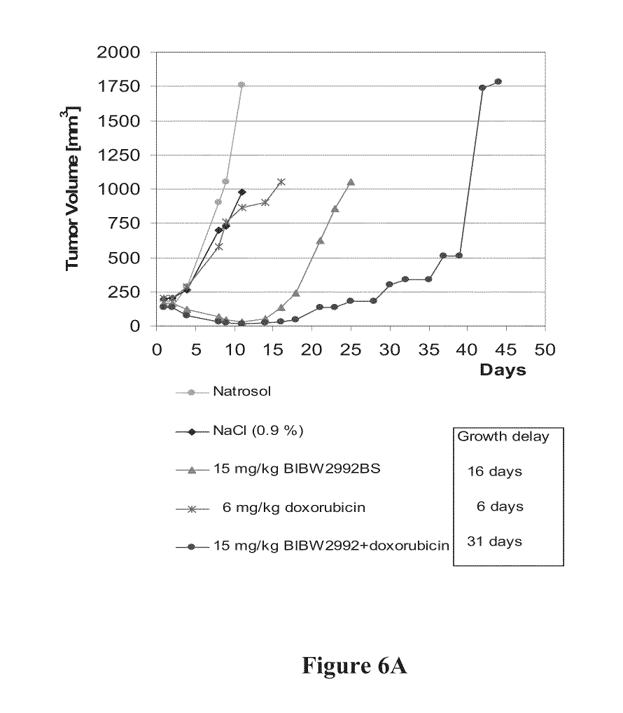

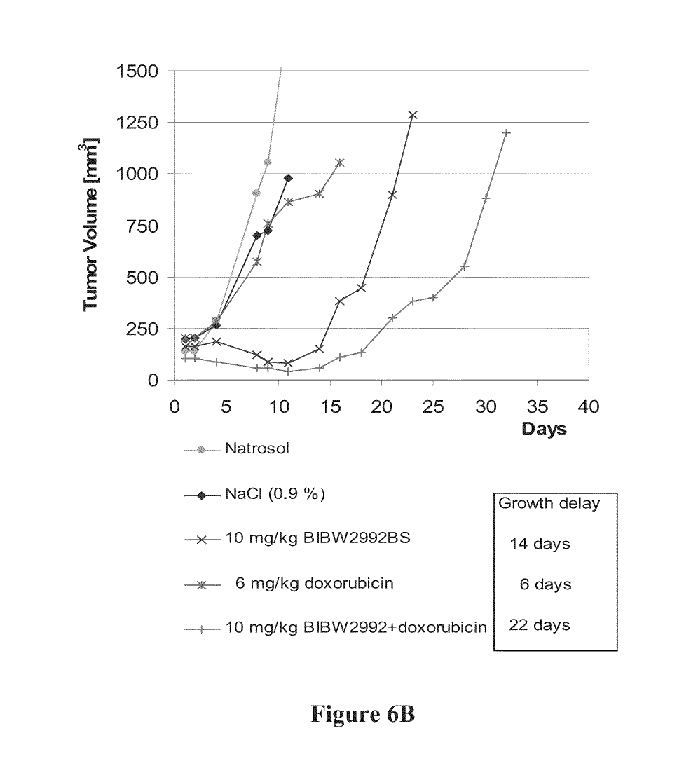

Results of an experiment comparing daily treatment (day 1-11) of MDA-453 breast xenografts in mice with BIBW2992 alone (two dosages of: 15 mg/kg; 10 mg/kg), doxorubicin alone (6 mg/kg) once weekly (day 1+8) and the combination of BIBW2992/doxorubicin (two dosages of: 15 mg/kg/6 mg/kg; 10 mg/kg/6 mg/kg) are shown in FIG. 6 (Appendix). This study clearly shows that the combination treatments with BIBW 2992 and doxorubicin resulted in better anti-tumor effects than either drug alone.

### Example 7

### Inhibition of 5-FU Combined with BIBW2992 in Anchorage Independent SKOV-3 Cell Assay

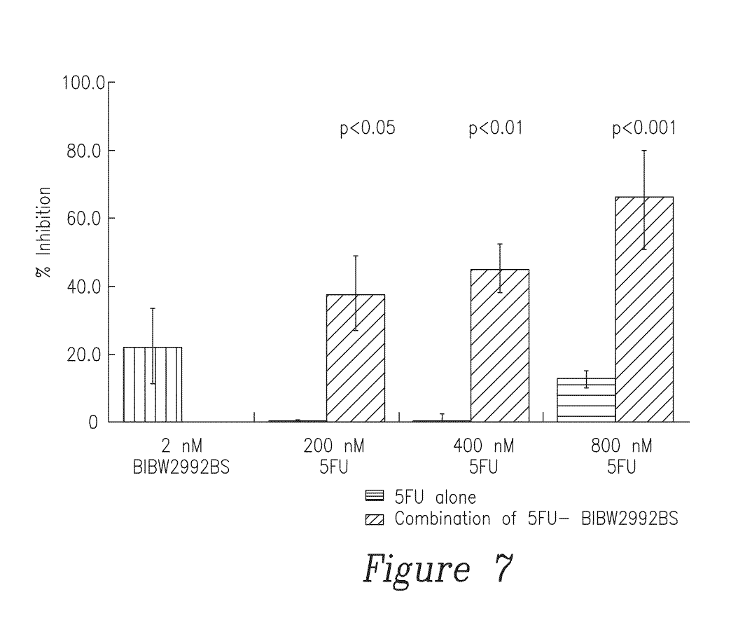

SKOV-3 cells are grown on a soft agar layer in the presence of 2 nM BIBW2992BS or three concentrations of 5FU (200 nM, 400 nM, 800 nM) or in combination of both. The results of the inhibitory effects are shown in FIG. 7 (Appendix).

No inhibition is observed in the presence of 200 and 400 nM 5FU whereas 2 nM is BIBW2992BS inhibits the cell growth by 20% and 800 nM 5FU by 13%. However, the inhibitory effect of the combination of both substances is significantly higher than 5FU or BIBW2992BS alone. The inhibitory effect of the combination is higher than the added effects of both.

The following Examples 8 to 28 contain as active substance a compound 1 of formula (I) or a small molecule compound (chemical entity) of chemotherapeutic agent 2.

### Example 8

### Coated Immediate-Release Tablets Containing 75 Mg of Active Substance by Dry-Granulation Process

### Composition:

1 tablet contains:

| | | |
|---|---|---|
| active substance | 75.0 | mg |
| calcium phosphate anhydrous | 108.0 | mg |
| corn starch | 35.5 | mg |
| polyvinylpyrrolidone | 10.0 | mg |
| magnesium stearate | 1.5 | mg |
| hydroxypropylmethylcellulose | 7.5 | mg |
| polyethylene glycol | 1.0 | mg |
| polydextrose | 5.0 | mg |
| talc | 1.0 | mg |
| pigments | 0.5 | mg |

| | |
|---|---|
| water (volatile) | *** |

| | |
|---|---|
| 245.0 | mg |

### Preparation:

The active substance is mixed with calcium phosphate, corn starch, polyvinylpyrrolidone, hydroxypropylmethylcellulose and half the specified amount of magnesium stearate. Ribbons are produced in a roller-compactor and these are then rubbed through a screen with a mesh size of 1.5 mm using a suitable machine and mixed with the rest of the magnesium stearate. This granulate is compressed in a tablet-making machine to form tablets of the desired shape.

Weight of core: 230 mg
Tablet shape: 9 mm round, bi-convex

The tablet cores are subsequently coated with an aqueous film-coat consisting essentially of hydroxypropylmethylcellulose, polyethylene glycol, polydextrose, talc and pigments.

Weight of coated tablet: 245 mg.

### Example 9

### Extended-Release Tablets Containing 100 Mg of Active Substance by Organic Granulation Process

1 tablet contains:

| | | |
|---|---|---|
| active substance | 100.0 | m [symbol] |
| lactose | 34.0 | mg |
| hydroxypropylmethylcellulose | 80 | mg |
| polyvinylpyrrolidone | 4.0 | mg |
| magnesium stearate | 2.0 | mg |

| | |
|---|---|
| ethanol (volatile) | *** |

| | |
|---|---|
| 220.0 | mg |

[symbol]

indicates data missing or illegible when filed

### Preparation:

The active substance, lactose and hydroxypropylmethylcellulose are mixed together and uniformly moistened with solution of the polyvinylpyrrolidone in ethanol. After the moist composition has been screened (2.0 mm mesh size) and dried in a rack-type drier at 50° C. it is screened again (1.5 mm mesh size) and the lubricant is added. The final blend is compressed to form tablets.
Weight of tablet: 220 mg
Tablet shape: 10 mm, flat-faced, with bevelled edges.

### Example 10

### Tablets Containing 150 Mg of Active Substance by Aqueous Granulation Process

1 tablet contains:

| | | |
|---|---|---|
| active substance | 150.0 | mg |
| powdered lactose | 98.0 | mg |
| corn starch | 40.0 | mg |
| colloidal silica | 1.0 | mg |
| polyvinylpyrrolidone | 10.0 | mg |
| magnesium stearate | 1.0 | mg |
| | 300.0 | mg |

### Preparation:

The active substance mixed with lactose, corn starch is moistened with a 20% aqueous polyvinylpyrrolidone solution and passed through a screen with a mesh size of 1.5 mm. The granules, dried at 45° C., are passed through the same screen again and mixed with the specified amount of magnesium stearate and colloidal silica. Tablets are pressed from the final blend.

Weight of tablet: 300 mg
Tablet shape: 14 mm×6.8 mm, oblong biconvex with embossement

### Example 11

### Hard Capsules Containing 150 Mg of Active Substance in Granules

### Composition:

1 capsule contains:

| | | |
|---|---|---|
| active substance | 150.0 | mg |
| microcrystalline cellulose | 80.0 | mg |
| lactose (spray-dried) | 87.0 | mg |
| colloidal silica | 10.0 | mg |
| | 320.0 | mg |

### Preparation:

The active substance is mixed with the excipients in a high-shear mixer, passed through a screen with a mesh size of 0.75 mm and homogeneously mixed using a suitable apparatus. The finished mixture is packed into size 1 hard gelatin capsules.

Capsule filling: 320 mg
Capsule shape: size 1, opaque hard capsule.

### Example 12

### Hard Capsules Containing 150 Mg of Active Substance as a Liquid Fill

### Composition:

1 capsule contains:

| | |
|---|---|
| active substance | 150.0 mg |
| groundnut oil | 300.0 mg |
| colloidal silica | 10.0 mg |
| | 460.0 mg |

### Preparation:

The active substance is dissolved in the excipient inside a homogenizer and the colloidal silica is added for adjustment of viscosity. The finished mixture is filled into size 1 hard gelatin capsules.

Capsule filling: 460 mg
Capsule shape: size 0, opaque hard capsules.

### Example 13

Suppositories Containing 150 Mg of Active Substance

### Composition:

1 suppository contains:

| | |
|---|---|
| active substance | 150.0 mg |
| polyethyleneglycol 1500 | 550.0 mg |
| polyethyleneglycol 6000 | 460.0 mg |
| polyoxyethylene sorbitan | 840.0 mg |
| monostearate | |
| | 2,000.0 mg |

### Preparation:

After the suppository mass has been melted the active substance is homogeneously suspended therein and the melt is poured into chilled moulds.

### Example 14

### Suspension Containing 50 Mg of Active Substance

### Composition:

100 ml of suspension contain:

| | | |
|---|---|---|
| active substance | 1.00 | g |
| carboxymethylcellulose-Na-salt | 0.10 | g |
| methyl p-hydroxybenzoate | 0.05 | g |
| propyl p-hydroxybenzoate | 0.01 | g |
| glucose | 10.00 | g |
| glycerol | 5.00 | g |
| 70% sorbitol solution | 20.00 | g |
| flavouring | 0.30 | g |
| dist. water | ad 100.0 | ml |

### Preparation:

The distilled water is heated to 70° C. The methyl and propyl p-hydroxybenzoates together with the glycerol and sodium salt of carboxymethylcellulose are dissolved therein with stirring. The solution is cooled to ambient temperature and the active substance is added and homogeneously dispersed therein with stirring. After the sugar, the sorbitol solution and the flavouring have been added and dissolved, the suspension is evacuated with stirring to eliminate air.

5 ml of suspension contain 50 mg of active substance.

### Example 15

### Ampoules Containing 10 Mg Active Substance

### Composition:

1 ampoule contains:

| active substance | 10.0 | mg |
|---|---|---|
| 0.01N hydrochloric acid. | q.s | |
| sodium chloride | q.s. | |
| double-distilled water | ad 2.0 | ml |

### Preparation:

The active substance is dissolved in the requisite amount of 0.01 N HCl, made isotonic with sodium chloride, filtered sterile and transferred into 2 ml ampoules with subsequent steam sterilization.

### Example 16

### Ampoules Containing 50 Mg of Active Substance

### Composition:

1 ampoule contains:

| active substance | 50.0 | mg |
|---|---|---|
| 0.01N hydrochloric acid | q.s. | |
| sodium chloride | q.s. | |
| double-distilled water | ad 10.0 | ml |

### Preparation:

The active substance is dissolved in the necessary amount of 0.01 N HCl, made isotonic with sodium chloride, filtered sterile and transferred into 10 ml ampoules with subsequent steam sterilization.

### Example 17

Capsules for powder inhalation containing 5 mg of active substance

### Composition:

1 capsule contains:

| | |
|---|---|
| active substance | 5.0 mg |
| lactose for inhalation | 15.0 mg |
| | 20.0 mg |

### Preparation:

The active substance is mixed with lactose for inhalation. The mixture is packed into capsules in a capsule-making machine (weight of the empty capsule approx. 50 mg).

weight of capsule: 70.0 mg
size of capsule 3

### Example 18

### Solution for Inhalation for Hand-Held Nebulisers Containing 2.5 Mg Active Substance

### Composition:

1 spray contains:

| | | |
|---|---|---|
| active substance | 2.500 | mg |
| benzalkonium chloride | 0.001 | mg |
| 1N hydrochloric acid q.s. | 2.500 | mg |
| ethanol/water (50/50 m/m) | ad 15.000 | mg |

### Preparation:

The active substance and benzalkonium chloride are dissolved in ethanol/water (50/50). The pH of the solution is adjusted with 1N hydrochloric acid. The resulting solution is filtered sterile and transferred into suitable containers for use in hand-held nebulisers (cartridges).

Contents of the container: 4.5 g

### Example 19

### Tablets Containing 150 Mg of Active Substance and a 150 Mg of a Second Active Substance by Aqueous Granulation Process

1 tablet contains:

| | |
|---|---|
| active substance | 150.0 mg |
| active substance 2 | 150.0 mg |
| powdered lactose | 98.0 mg |
| corn starch | 40.0 mg |
| colloidal silica | 1.0 mg |
| polyvinylpyrrolidone | 10.0 mg |
| magnesium stearate | 1.0 mg |
| | 450.0 mg |

### Preparation:

The both active substances are mixed with lactose, corn starch is moistened with a 20% aqueous polyvinylpyrrolidone solution and passed through a screen with a mesh size of 1.5 mm. The granules, dried at 45° C., are passed through the same screen again and mixed with the specified amount of magnesium stearate and colloidal silica. Tablets are pressed from the final blend.

Weight of tablet: 450 mg
Tablet shape: 15.0 mm×7.0 mm, oval biconvex with embossement

### Example 20

### Hard Capsules Containing 150 Mg of Active Substance in Coated Pellets and 150 Mg of a Second Active in Coated Pellets

### Composition:

1 capsule contains:

| | |
|---|---|
| active substance | 150.0 mg |
| active substance 2 | 150.0 mg |
| powdered lactose | 98.0 mg |
| corn starch | 40.0 mg |
| polyvinylpyrrolidone | 10.0 mg |
| hydroxypropylmethylcellulose | 7.5 mg |
| polyethylene glycol | 1.0 mg |
| polydextrose | 5.0 mg |
| talc | 1.0 mg |
| water (volatile) | *** |
| | 462.50 mg |

### Preparation:

The active substances are separately extruded with half of the lactose and the corn starch by a wet-extrusion process and rounded in a spheronizer to pellet. Each fraction is dryed in a fluid-bed dryer/coater and subsequently coated with half of a solution of the other excipients. The dryed pellets are homogeneously mixed and packed into size 0 hard capsules.

Capsule filling: 462.5 mg
Capsule shape: size 0, opaque hard capsule.

1. A method of treating cancer comprising administering to a patient therapeutically effective amounts of: 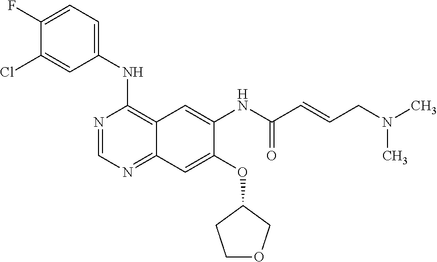 [SMILES: CN(C)C/C=C/C(=O)Nc1cc2c(Nc3ccc(F)c(Cl)c3)ncnc2cc1O[C@H]1CCOC1]
4-[(3-chloro-4-fluorophenyl)amino]-6-{[4-(N,N-dimethylamino)-1-oxo-2-buten-1-yl]amino}-7-((S)-tetrahydrofuran-3-yloxy)-quinazoline
or a pharmacologically acceptable acid addition salt thereof,
and cetuximab;
wherein cancer to be treated is selected from
Head and neck tumours: SCC, AC, transitional cell cancers, mucoepidermoid cancers, undifferentiated carcinomas;
Colorectal cancers: AC, including hereditary forms of AC, carcinoid, sarcoma;
Pancreatic cancers: AC, including ductal and acinary cancers, papillary, adenosquamous, undifferentiated, tumours of the endocrine pancreas;
Breast cancers: AC, including invasive ductal, lobular and medullary cancers, tubular, mucinous cancers, Paget-carcinoma, inflammatory carcinoma, ductal and lobular carcinoma in situ;
Prostate cancers: AC, small cell, SCC;
Gastric cancers: AC, adenosquamous, anaplastic;
Ovarian cancer; and
Non-small cell lung cancers (NSCLC): SCC, spindle cell carcinoma, AC, bronchioalveolar carcinoma, large cell NSCLC, clear cell NSCLC.

2. The method according to claim 1, wherein the patient is a pre-selected cancer patient shown to carry a tumor harboring an activating EGFR mutation.

3. The method according to claim 2, wherein the EGFR mutation is selected from the group consisting of the L858R point mutation, deletion/insertion mutations in the ELREA sequence, the T790M point mutation in exon 20, and double mutations such as the combined L858R/T790M mutation.

4. The method according to claim 1, wherein the patient is a pre-selected cancer patient shown to carry a tumor harboring an activating HER2 mutation.

5. The method according to claim 4, wherein the HER2 mutation is the M774_A775insAYVM mutation.

6. The method according to claim 1, 3 or 5, wherein the cancer is selected from Head and neck tumours: SCC, AC, transitional cell cancers, mucoepidermoid cancers and undifferentiated carcinomas.

7. The method according to claim 1, 3 or 5, wherein the cancer is selected from Colorectal cancers: AC, hereditary forms of AC, carcinoid and sarcoma.

8. The method according to claim 1, 3 or 5, wherein the cancer is selected from Non-small cell lung cancers (NSCLC): SCC, spindle cell carcinoma, AC, bronchioalveolar carcinoma, large cell NSCLC and clear cell NSCLC.
